(19) United States
(12) Patent Application Publication
(10) Pub. No.: US 2019/0054031 A1
(43) Pub. Date: Feb. 21, 2019

(54) OVERDOSE PROTECTION AND ABUSE DETERRENT IMMEDIATE RELEASE DRUG FORMULATION

(71) Applicant: KASHIV PHARMA LLC, Bridgewater, NJ (US)

(72) Inventors: Navnit H. Shah, Clifton, NJ (US); Wantanee Phuapradit, Montville, NJ (US); Dipen Desai, Whippany, NJ (US); Siva Ram Kiran Vaka, Piscataway, NJ (US); Kanji Meghpara, Morris Plains, NJ (US)

(73) Assignee: KASHIV PHARMA LLC, Bridgewater, NJ (US)

(21) Appl. No.: 15/764,464

(22) PCT Filed: Sep. 30, 2016
(86) PCT No.: PCT/US2016/055022

§ 371 Date: Mar. 29, 2018

Related U.S. Application Data
(60) Provisional application No. 62/234,881, filed on Sep. 30, 2015.

(51) Int. Cl. A61K 9/50; A61K 9/48; A61K 31/485; A61K 9/20; A61P 25/36

(52) U.S. Cl. CPC A61K 9/5073; A61P 25/36; A61K 9/4866; A61K 31/485; A61K 9/4858; A61K 9/5047; A61K 9/5015; A61K 9/501; A61K 9/2081; A61K 9/2054; A61K 9/2009; A61K 9/2013; A61K 9/2027; A61K 9/2018; A61K 9/5084; A61K 9/2086; A61K 9/485

(57) ABSTRACT

The presently disclosed subject matter provides a solid immediate release pharmaceutical multi-particulate dosage form containing at least two different populations of particulates. In certain embodiments, the immediate release pharmaceutical dosage forms contain at least three different populations of multi-particulates. Each population of particulates is designed for a specific function to accomplish the desired combination of abuse deterrence and overdose protection.

This application claims priority to U.S. Provisional Patent Application No. 62/234,881, filed Sep. 30, 2015, the disclosure of which is incorporated by reference herein in its entirety.

### 1. FIELD OF THE INVENTION

The present invention relates to immediate release pharmaceutical dosage forms with abuse deterrent (AD) and overdose protection (ODP) properties/features, and processes of manufacture.

### 2. BACKGROUND

Governmental reports state that prescription drug abuse is the fastest growing drug problem in the United States, and a survey indicated that nearly one-third of people age 12 and above who used drugs illicitly for the first time in 2009 began by the nonmedical use of a prescription drug. For example, opioid analgesics can be abused by: swallowing whole in excessive quantities; crushing and swallowing; crushing and inhaling nasally (“snorting”); crushing and smoking; or crushing, dissolving, and injecting the prescription drug.

Abuse can also involve some physical or mechanical manipulation of a dosage form so that larger amounts of immediately available drug can be taken orally, nasally, or by intravenous injection. Reports of overdosing and death from prescription pain products rose sharply in the early 2000s. For example, among opioid dosage forms, immediate release oxycodone is the third most prone to overdose.

The U.S. Food and Drug Administration (FDA) describes the science of abuse deterrence as relatively new and rapidly evolving. In April 2015, the FDA published a draft guidance document for the evaluation and labeling of abuse-deterrent opioid products. Categories of abuse-deterrent formulations were described as:
1. Physical barriers to prevent chewing, crushing, cutting, grating or grinding, and chemical barriers to resist extraction of the active ingredient with common solvents such as water, alcohol, and organic liquids;
2. Agonist/antagonist combinations that interfere with, reduce, or defeat the euphoria associated with abuse;
3. Aversion, by incorporating a substance that produces an unpleasant effect when the dosage form is altered before ingestion, or is ingested in a high dose;
4. Delivery systems that provide abuse resistance through release characteristic design or a mode of administration;
5. New molecular entities and prodrugs that lack opioid activity until acted upon in the gastrointestinal system;
6. Combinations of two or more of the foregoing; and
7. Novel approaches not captured by the other categories.

In March 2016, the FDA published a guidance document describing general procedures for developing and evaluating abuse deterrence of generic solid oral opioid products formulated to incorporate physical or chemical barriers, agonists/antagonists, aversive agents, or combinations of these technologies. The FDA recommends the following evaluations, involving all potential routes of abuse, of the abuse deterrence of generic solid oral opioid drug products:
1. Injection (parenteral route)—evaluate the extractability and syringeability of intact and mechanically manipulated products.
2. Ingestion (oral route)—evaluate extractability, dissolution, and where applicable, the rate and extent of a product's absorption for intact and mechanically or chemically manipulated products.
3. Insufflation (nasal route)—evaluate nasal availability and likability of mechanically manipulated and insufflated products.
4. Smoking (inhalation route)—evaluate the ability to sublimate intact and mechanically or chemically manipulated products.

The FDA further describes mechanical manipulation, with and without thermal pretreatment (e.g., freezing at −20° C., or heating), as involving cutting, grating, and milling.

A few abuse-resistant opioid products are currently approved for marketing, including OXYCONTIN® (oxycodone hydrochloride extended release tablets), XTAMPZA™ ER (oxycodone hydrochloride ER), TARGINIQ® (oxycodone HCl and naloxone HCl), and EMBEDA® (morphine sulfate and naltrexone hydrochloride). Other products, such as OXAYDO® (oxycodone hydrochloride IR tablets), SUBOXONE® (buprenorphine and naloxone) and OPANA ER® (oxymorphone), also purport to have abuse deterrent properties but do not have a formal claim on the label. As noted by the FDA in their 2015 guidelines, most abuse-deterrent technologies have not yet proven successful at deterring the most common form of abuse: swallowing a number of intact capsules or tablets.

A need, therefore, remains for improved formulations that make it difficult, if not impossible, for individuals to abuse or misuse opioids, not only by snorting and/or extraction of drug, but also by ingesting multiple doses. In particular, new formulations are needed that can be used with immediate release pharmaceutical products. There is also a need for improved formulations that reduce or prevent the effects of overdose, whether intentional or unintentional (e.g., accidental). Such formulations should combine overdose protection and abuse deterrence in a single dosage form and thereby address multiple health-related concerns, especially regarding habit-forming opioid compounds, for which there is a high propensity for abuse and overdose. These dosage forms must also allow the active pharmaceutical ingredient to be soluble in the gastrointestinal tract and have the desired pharmacological activity. In the case of opioids, the pharmacological activity would be, for example, an analgesic effect.

### 3. SUMMARY OF THE INVENTION

The presently disclosed subject matter provides an abuse deterrent and/or overdose resistant immediate release pharmaceutical particulate or multi-particulate dosage form containing at least two different populations of particulates.

In certain embodiments, included in the scope of the invention is a solid immediate release (IR) multi-particulate dosage form with abuse deterrent and overdose protection properties comprising a first population of particulates comprising a therapeutically effective amount of at least one active agent (e.g., an opioid) embedded in a polymer matrix, at least one functional coat (e.g., FC 0, FC 1, FC 2 layers), and an over coat. In certain embodiments, FC 1 layer comprises a nonionic pH-independent polymer (nonionic polymer) insoluble in physiological fluids and/or organic solvents, and a cationic pH-dependent polymer (cationic polymer) that acts as a pore former at a pH of less than about 5.0. In certain embodiments, the over coat comprises a nonionic water-soluble polymer. In certain embodiments, a second population of particulates comprises an alkaline agent. In certain embodiments, the second population of particulates comprises an alkaline agent and a pH-stabilizing agent. In certain embodiments, the alkaline agent raises the gastric pH when three or more dosage units are ingested, and the pH-stabilizing agent maintains the elevated pH for a finite time.

In certain embodiments, the abuse deterrent properties comprise reduction in abuse potential by, for example, smoking, intranasal and/or intravenous routes, and/or orally upon ingesting three or more intact tablets together (i.e., ODP).

In certain embodiments, the ODP properties comprise reduction in opioid release to less than about 50% at 30 minutes when three or more units of the dosage form are consumed.

In certain embodiments, the abuse deterrent properties comprise resistance to syringeability, wherein less than 10% of the opioid is available in a syringeable form, e.g., less than 10% of the opioid provided in a dosage form can be extracted, after grinding or crushing followed by dissolution/suspension in a liquid, as a syringeable liquid.

In certain embodiments, abuse deterrent properties comprise resistance to grinding/crushing, wherein grinding or crushing of the dosage form provides more than 50% of particulates in the size range of 250-500 μm or greater.

In certain embodiments, the abuse deterrent elements enhance the ODP properties of the dosage form.

In certain embodiments, the ODP elements enhance abuse deterrent properties of the dosage form.

### 4. BRIEF DESCRIPTION OF THE FIGURES

FIG. 1 depicts a schematic representation of an Active Granule according to certain embodiments.

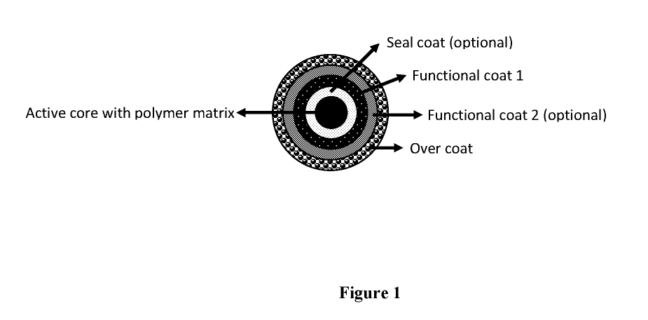

FIG. 2 shows the effects of a single unit versus five units on percentage of oxycodone released in (initial) pH 1.6, wherein the seal coated Active Pellets are further coated with a functional coat comprising OPADRY® CA and EUDRAGIT® E PO at a ratio of 60:40. Each unit represents a 30 mg oxycodone hydrochloride dosage form.

FIG. 3 shows the effects of a single unit versus two units, three units, and five units on the percentage of oxycodone released in (initial) pH 1.6, wherein the seal coated Active Pellets are further coated with a functional coat comprising OPADRY® CA and EUDRAGIT® E PO at a ratio of 80:20. Each unit represents a 30 mg oxycodone hydrochloride dosage form.

FIG. 4 shows a dissolution profile of oxycodone hydrochloride from oxycodone hydrochloride tablets (i.e., tablets of the invention; “OXY”; 15 mg) and ROXICODONE® tablets (“Roxi”; 15 mg), one unit versus three units and six units, in a two-stage dissolution method: the first stage is in pH 1.6 for 30 minutes, followed by a second stage in pH 6.8 for 120 minutes.

FIG. 5 shows the effect of the number of oxycodone hydrochloride tablets (one, three, and six tablets) on pH with time.

FIG. 6 shows a dissolution profile of hydromorphone hydrochloride from hydromorphone hydrochloride tablets (8 mg), one unit versus three units and six units, in a two-stage dissolution method: the first stage is in pH 1.6 for 30 minutes, followed by a second stage in pH 6.8 for 150 minutes.

FIG. 7a shows particle size distribution (PSD) and active pharmaceutical ingredient (API) distribution across sieve fractions of manipulated granules (i.e., granules of the invention; equivalent to 5 mg and 15 mg oxycodone hydrochloride tablet strengths) using a mortar and pestle (MP) and an electric coffee grinder (CG).

FIG. 7b shows PSD and API distribution across sieve fractions of manipulated granules (equivalent to 8 mg hydromorphone hydrochloride tablet strength) using MP and CG.

FIG. 7c shows PSD and API distribution across sieve fractions of manipulated granules (10 mg hydrocodone bitartrate granules) using MP and CG.

FIG. 8a shows PSD and API distribution across sieve fractions of manipulated ROXICODONE® tablets (15 mg strength) and oxycodone tablets (i.e., tablets of the invention; 15 mg and 5 mg strengths) using MP and CG.

FIG. 8b shows PSD and API distribution across sieve fractions of manipulated hydromorphone hydrochloride tablets (8 mg strength) using MP and CG.

FIG. 9 shows gelling behavior of ROXICODONE® (RLD) (15 mg strength) and oxycodone hydrochloride tablets (i.e., tablets of the invention; 5 and 15 mg strengths) when manipulated and incubated in water at ambient conditions for syringeability studies. The image depicts (left to right) 15 mg and 5 mg oxycodone (tablet of the invention), and RLD, both before withdrawal (triplet at left) and after withdrawal (triplet at right).

FIG. 10 shows percent volume of supernatant liquid withdrawn into a syringe after 30 minute incubation with water at ambient conditions after manipulation of ROXICODONE® tablets (LD; 15 mg strength), oxycodone hydrochloride tablets (Oxy; 15 and 5 mg strengths), and hydromorphone hydrochloride tablets (8 mg strength).

FIG. 11 shows percentage of opioid present in supernatant liquid withdrawn into a syringe after 30 minute incubation with water at ambient conditions after manipulation of ROXICODONE® tablets (LD; 15 mg strength), oxycodone hydrochloride tablets (Oxy; 15 and 5 mg strengths), and hydromorphone hydrochloride tablets (8 mg strength).

### 5. DETAILED DESCRIPTION

To date, there remains a need for improved immediate release pharmaceutical dosage forms that make it difficult, if not impossible, for individuals to take the dosage forms in a manner other than intended by the manufacturer. In certain embodiments, the present invention provides improved solid oral immediate release pharmaceutical particulate and multi-particulate dosage forms containing at least one population of particulates, e.g., particulates comprising an active agent (e.g., an opioid). In certain embodiments, the present invention provides improved solid oral immediate release pharmaceutical multi-particulate dosage forms containing at least two populations of particulates, e.g., (1) Active Particulates containing an opioid(s), and (2) Triggering Particulates containing an alkaline agent(s) and/or a pH-stabilizing agent(s). In certain embodiments, the immediate release pharmaceutical multi-particulate dosage forms contain at least three different populations of particulates. In certain embodiments, the immediate release pharmaceutical multi-particulate dosage forms contain at least four, at least five, or at least six different populations of particulates. In certain embodiments, the Active Particulates comprise an opioid(s), alkaline agent(s), and/or a pH-stabilizing agent(s); in certain embodiments, the alkaline agent(s) and/or pH-stabilizing agent(s) can be covering/surrounding the Active Particulates. Each population of particulates is designed for a specific function to accomplish the desired combination of abuse deterrence and overdose protection.

In certain embodiments, the immediate release pharmaceutical dosage forms contain an Active Particulate population (i.e., Active Granules or Active Pellets), which is a crush-resistant particulate population comprising an active agent and at least a first functional coat layer (e.g., FC 1) that allows the release of the active agent in an aqueous or nonaqueous environment with a pH of up to about 5.0, providing overdose protection (ODP). In certain embodiments, the Active Particulates can further include a seal coat between the core (e.g., the polymer matrix of an Active Granule) and the first functional coat layer. In certain embodiments, the Active Particulates can further include a second functional coat layer (e.g., FC 2) on top of FC 1. In certain embodiments, the Active Particulates can include an additional functional coat layer (referred to as FC 0) between the seal coat (or the core) and FC 1. In certain embodiments, FC 0 and FC 2 can further enhance the ODP features of the Active Particulates in the event of an overdose (e.g., administration/consumption of three or more dosage units). In certain embodiments, FC 0 and/or FC 2 aid FC 1 in preventing or slowing release of the active agent from the Active Particulate in an aqueous or nonaqueous environment with a pH above about 5.0. In certain embodiments, the Active Particulates can further include an over coat that aids in maintaining the controlled release of active agent. In certain embodiments, the over coat prevents/reduces the interaction of EUDRAGIT® E PO present in the functional coat layer(s) (e.g., FC 1, or, when present, FC 2) with the alkaline agent present in the Triggering Particulates in the dosage form to maintain the controlled release of the active agent.

In certain embodiments, Active Particulates contain an opioid(s) as the active agent (Opioid Particulates).

In certain embodiments, the dosage form contains a Triggering Particulate (e.g., Triggering Granule) containing an alkaline agent that increases the pH of the aqueous or nonaqueous solution to above about pH 5.0 in the presence of three or more dosage units. The Triggering Particulate can also contain a pH-stabilizing agent that maintains the increased pH above about 5.0 for up to five minutes, up to ten minutes, up to 15 minutes, up to 30 minutes, up to 45 minutes, up to one hour, up to 1.5 hours, or up to two hours or more. In certain embodiments, the increase in pH above about 5.0 reduces the dissolution of the functional coat (e.g., one or more functional coat layers), and thereby prevents or slows the release of the active agent from the Active Particulates.

In certain embodiments, the immediate release pharmaceutical dosage forms comprise a Viscosity Enhancing Particulate population (e.g., Viscosity Enhancing Granules) containing a viscosity-building polymer(s) that increases the viscosity of the aqueous or nonaqueous solution if tampered with or taken in doses above those prescribed or in a manner inconsistent with the manufacturer's instructions.

In certain embodiments, the pharmaceutical compounds for use in the present invention are those at risk for accidental (e.g., unintentional) or intentional overdose via, for example, the oral route, or misuse via, for example, the oral/intravenous/nasal/smoking route(s). In certain embodiments, the active agent is an opioid.

### 5.1. Definitions

The terms used in this specification generally have their ordinary meanings in the art, within the context of this invention and in the specific context where each term is used. Certain terms are discussed below, or elsewhere in the specification, to provide additional guidance to the practitioner in describing the compositions and methods of the invention and how to make and use them.

As used herein, the use of the word “a” or “an” when used in conjunction with the term “comprising” in the claims and/or the specification can mean “one,” but it is also consistent with the meaning of “one or more,” “at least one,” and “one or more than one.” Still further, the terms “having,” “including,” “containing” and “comprising” are interchangeable, and one of skill in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 15%, up to 10%, up to 5%, or up to 1% of a given value. Alternatively, particularly with respect to biological systems or processes, the term can mean within an order of magnitude, preferably within five-fold, and more preferably within two-fold, of a value.

The term “active agent,” “drug,” “compound,” “active pharmaceutical ingredient,” or “API” refers to a pharmaceutically active substance which includes, without limitation, drugs susceptible to abuse and/or overdose. In certain embodiments, the active agent is an opioid analgesic.

The term “opioid” or “opioid analgesic” includes single compounds and a mixture of compounds selected from the group of opioids that provide, e.g., an analgesic effect. For example, opioids can include, without limitation, an opioid agonist, a mixed opioid agonist-antagonist, or a partial opioid agonist. In certain embodiments, the opioid can be a stereoisomer, ether, salt, hydrate or solvate thereof. The terms opioid and opioid analgesic are also meant to encompass the use of all such possible forms as well as their racemic and resolved forms thereof, and all tautomers as well. The term “racemic” refers to a mixture of equal parts of enantiomers.

The term “immediate release” or “IR” refers to dosage forms that are formulated to allow the drug to dissolve in the gastrointestinal contents/fluids with no intention of delaying or prolonging the dissolution or absorption of the drug when taken as prescribed or in a manner consistent with manufacturer's instructions.

The term “extended release” or “ER” refers to dosage forms that are formulated to allow the drug to be available over a greater period of time after administration, thereby allowing a reduction in dosing frequency, as compared to a drug presented as a conventional dosage form (e.g., immediate release).

The term “particulate” refers to a discrete, small, repetitive unit of particles, granules, or pellets that include at least one excipient and, optionally, an active agent (e.g., an opioid).

The term “multi-particulate” refers to at least two different populations of particulates.

The term “dosage form” refers to an oral particulate solid drug delivery system that, in the present technology, includes at least one or two populations of particulates.

The term “dosage unit” refers to a single tablet (e.g., tablet, tablet-in-tablet, bilayer tablet, multilayer tablet, etc.), capsule, pill, or other solid dosage form.

The term “coat” refers to a coating, layer, membrane, film, etc. applied to a surface, and, in certain embodiments, can partially, substantially, or completely surround, envelop, cover, enclose, or encase the surface of a particulate, granule, drug, dosage unit, or the like to which it is applied. For example, a coat may cover portions of the surface to which it is applied, e.g., as a partial layer, partial coating, partial membrane, or partial film, or the coat may completely cover the surface to which it is applied.

The terms “acid labile coat” or “functional coat” (or “coatings”) refer to a coat comprising a component(s) that will dissolve or degrade (partially or completely) in an acidic environment (e.g., in a solution with an acidic pH). In certain embodiments, the acidic pH may be, for example, below about 7.0, below about 6.0, below about 5.0, below about 4.0, below about 3.0, or below about 2.0, or below about 1.0. Typically, the pH at which an acid labile coat/functional coat of the present invention will dissolve is in the normal physiological pH (e.g., the range of normal physiological pH values) of the stomach, such as from about 1.0 to about 5.0, from about 1.0 to about 4.0, or from about 2.0 to about 3.0. Typically, the acid labile coat/functional coat dissolves or degrades more slowly, or to only a small extent, when present in a solution with a pH that is considered not acidic (e.g., nonacidic and/or less acidic; e.g., at a pH above about 5.0, above about 6.0, or above about 7.0). It will be understood that the acid labile coat/functional coat can be prepared and designed to dissolve or degrade (partially or substantially) within any desired pH range, and to not dissolve or degrade (partially or substantially) within any desired pH range. For example, the acid labile coat/functional coat can be designed to dissolve at any pH, e.g., below about 5.0; above that level, dissolution is inhibited, reduced or slowed. As the pH increases, the dissolution/degradation may slow further, and may stop nearly completely. The acid labile coat/functional coat affects the rate of release, in vitro or in vivo, of an active drug(s), e.g., an opioid(s). Such coatings or coats are sometimes referred to as “rate-limiting” or “rate-controlling”; the particular polymer(s) responsible for affecting the rate of release in the coating or coat can also be referred to as “rate-limiting” or “rate-controlling.” An acid labile coat/functional coat can comprise one or more functional coat layers.

The term “alkaline agent” may be used to refer to an excipient that acts to increase the pH of, e.g., the gastric fluid (e.g., roughly pH 1.2-4.5) to a pH greater than about 5.0. For example, alkaline agent may refer to substances that are capable of increasing the pH to greater than 4.5, greater than 5.0, greater than 5.5, etc. It also refers to basic substances and substances that can convert an acidic environment to a less acidic or a basic environment. Typically, these agents, when present in a sufficient amount, are able to raise the pH of the stomach to beyond physiological levels and thereby prevent, reduce, or inhibit dissolution of an acid labile substance or coat. Examples of alkaline agents include: aluminum hydroxide, sodium hydroxide, potassium hydroxide, calcium hydroxide, magnesium hydroxide, aluminum oxide, sodium oxide, potassium oxide, calcium oxide, magnesium oxide, calcium carbonate, sodium carbonate, potassium bicarbonate, sodium bicarbonate, ammonia, tertiary sodium phosphate, diethanolamine, ethylenediamine, N-methylglucamine, L-lysine, and combinations thereof.

The term “pH-stabilizing agent” refers to salts of weak acids/weak bases that act to maintain or stabilize the elevated pH of gastric fluid caused by the alkaline agent. For example, a pH-stabilizing agent(s) maintains the pH of the gastric fluid at a pH greater than 5.0 for a finite time.

The term “viscosity-building polymer” as used herein refers to a polymer or group of polymers that increase the viscosity of a solution if the dosage form is tampered with or taken in doses above those prescribed or in a manner inconsistent with the manufacturer's instructions.

The term “nonionic polymer” refers to a nonionic pH-independent polymer.

The term “water-insoluble nonionic polymer” refers to a nonionic pH-independent polymer generally insoluble in water, physiological fluids, and ethanol.

The term “water-soluble nonionic polymer” refers to a nonionic pH-independent polymer generally soluble in water, physiological fluids, and ethanol.

The term “cationic polymer” refers to a cationic pH-dependent polymer, generally soluble in a particular pH range, e.g., gastric fluid or simulated gastric fluid (SGF) (e.g., a polymer, containing one or more cationic groups, soluble in, e.g., gastric fluid or SGF).

The term “mini-tablet” refers to a tablet with a diameter equal to or smaller than 4 mm. They can be filled into a capsule or compressed into a larger tablet.

The term “abuse-deterrent formulation,” “abuse-deterrent composition,” “abuse-resistant formulation,” “abuse-resistant composition,” or “ADF” are used interchangeably to refer to a dosage form that reduces the potential for abuse but delivers a therapeutically effective dose when administered as directed. For example, these terms refer to a dosage form that can be at least resistant, with or without heat treatment or freezing, to crushing, grinding, melting, cutting, extracting, dose dumping (e.g., alcohol dose dumping), and solubilizing for injection purposes. Improper administration includes, without limitation, tampering with the dosage form and/or administering the drug by any route other than that instructed. For example, and without limitation, improper administration includes snorting after grinding, administration after heat treatment, oral administration after crushing, or parenteral administration after extraction with a solvent such as water, ethanol, isopropanol, acetone, acetic acid, vinegar, carbonated beverages, and the like, and combinations thereof.

The term “abuse” means the intentional, nontherapeutic use of a dosage form or active agent, to achieve a desirable psychological or physiological effect. For example, these terms refer to tampering with the dosage form and/or administering the drug in a manner inconsistent with the manufacturer's instructions. Methods of tampering or abuse include, but are not limited to, crushing, grinding, melting, cutting, heating, freezing, extracting, dose dumping (e.g., alcohol dose dumping), and solubilizing for injection purposes.

The term “in a manner inconsistent with the manufacturer's instructions” is meant to include, but is not limited to, consuming amounts greater than amounts described on the label or prescribed by a licensed physician, and/or altering by any means (e.g., crushing, breaking, milling, melting, separating, etc.) the dosage forms such that the active agent maybe crushed, ground, melted, cut, extracted, dose dumped (e.g., alcohol dose dumping), and/or solubilized for injection purposes.

The term “syringeability” refers, for example, to the ability of an agent (e.g., an opioid) to be extracted from a product formulation or dosage form into a syringe, i.e., the agent is in a syringeable form. For example, a solid dosage form may be dissolved/suspended in water, and an agent present in the dosage form can be extracted from the resulting liquid into a syringe in the form of a syringeable liquid.

The term “available in syringeable form,” as used herein, refers to availability of an agent (e.g., an opioid) to be extracted into a syringe from a solution/suspension of a solid dosage form. The amount or percentage of such extracted agent could be termed as the amount or percentage available in syringeable form, or available as a syringeable liquid, or the like.

The term “crush resistant” or “resistant to crushing” means, for example, a granule or particulate (e.g., an Active Granule) that may deform but does not break into powder form when pressure greater than 500 N is applied, when using a suitable hardness tester. Such resistance to crushing deters the abuse of the dosage form.

The term “grinding” refers to a process of reducing, or attempting to reduce, one or more tablets into small fragments, e.g., in the form of powder, following a specific grinding pattern (e.g., two minutes grinding/one minute rest/two minutes grinding) using, for example, an electrical grinding means (e.g., coffee grinder or IKA grinder).

The terms “resistant to alcohol extraction” and “resistant to alcohol dose-dumping” are used to refer to two or more dosage units (e.g., any form(s) of tablets or capsules) that at least fulfill the condition that in vitro dissolution, characterized by the percentage of active agent released at, e.g., 30 minutes or 60 minutes of dissolution, when measured in a USP Apparatus 1 (basket) at 100 rpm in 900 ml simulated gastric fluid comprising 40% ethanol at 37° C., deviates no more than 20% from the corresponding in vitro dissolution measured at the same time point in the same apparatus at the same speed in 900 ml SGF without ethanol at 37° C. Such resistance to alcohol dose dumping deters the abuse of the dosage form.

The term “overdose protection” or “ODP” refers to an oral dosage form that reduces the potential for overdose but delivers a therapeutically effective dose when administered as directed or ordered by a licensed physician.

The term “overdose” refers to the administration of the dosage form in amounts or doses above those considered therapeutic (e.g., three or more dosage units; more than two dosage units); in a manner inconsistent with manufacturer's instructions; or in a manner not prescribed. Overdose can be intentional or unintentional (e.g., accidental).

As used herein, use of phrases such as “decreased,” “reduced,” “diminished,” or “lowered” is meant to include at least a 10% change in, e.g., the release of an active agent, with greater percentage changes being preferred for reduction in abuse potential and overdose potential. For example, but without limitation, the change may be greater than 25%, 35%, 45%, 55%, 65%, 75%, 85%, 95%, 96%, 97%, 98%, 99%, or increments therein.

### 5.2. Active Particulates

The Active Particulates contain the active agent. In certain embodiments, the Active Particulates are Active Granules, Active Pellets, or a combination thereof. In certain embodiments, the Active Particulates are Active Granules. In certain embodiments, the Active Granules can include a polymer matrix that in some embodiments may include an active agent, a hydrophilic polyethylene oxide (PEO) polymer, a cationic and/or a nonionic polymer, an antioxidant, a plasticizer, and/or a surfactant. The polymer matrix of, e.g., the Active Granules containing the active agent can be directly coated/surrounded by a seal coat. In certain embodiments, the seal coat can be made with a water-soluble nonionic polymer. In certain embodiments, the seal coat is optional. In certain embodiments, the polymer matrix core (in absence of a seal coat)), or the seal coat (when present over the polymer matrix core) is surrounded by one or more functional coat layers (e.g., FC 0, FC 1, FC 2). In certain embodiments, the polymer matrix, or the seal coat covering the polymer matrix is directly covered by at least one functional coat layer (e.g., FC 1). In certain embodiments, one or more functional coats can include a water-insoluble nonionic polymer, as well as a cationic polymer that behaves as a pore former at pH below about 5.0. In certain embodiments, the Active Particulates comprising FC 1 may further comprise FC 0, located between the polymer matrix (or seal coat) and FC 1. In certain embodiments, the Active Particulates comprising FC 1 may further comprise FC 2, coated over FC 1. In certain embodiments, FC 0 and/or FC 2 contain a cationic polymer and, optionally, a nonionic polymer. In certain embodiments, the Active Particulates further include an over coat that contains a water-soluble nonionic polymer and covers the one or more functional coat layer(s), e.g., surrounds the outermost layer.

In certain embodiments of Active Particulates, each of FC 0, FC 1, and/or FC 2 accomplishes the role of overdose protection coupled with the alkaline agent(s) and, optionally, pH-stabilizing agent(s) contained in, e.g., one of the other particulates (i.e., Triggering Particulates, as described herein) present in the ADF-ODP dosage form (tablets, capsules, etc.). In certain embodiments, FC 0 and/or FC 2 may provide enhanced ODP, in addition to that provided by FC 1, when coupled with the alkaline agent(s) and/or pH-stabilizing agent(s) contained in the Triggering Particulates.

### 5.2.1. Active Agents

In certain embodiments, the Active Particulates contain at least one active agent, e.g., an opioid. In certain embodiments, different populations of Active Particulates contain different active agents. In certain embodiments, the active agent has a solubility of greater than about 100 microgram/ml of physiological fluids (e.g., GI fluids, SGF).

The Active Particulates can be coated with at least one functional coat layer (e.g., FC 1). In certain embodiments, FC 1 includes a nonionic polymer that is insoluble in water and a cationic polymer that behaves as a pore former at a pH from about 1.2 to about 4.5 or 5.0 and is insoluble in fluids with a pH above about 5.0 (e.g., at a pH of about 5.0 or greater). Surprisingly, it has been found that a functional coat (e.g., at least one functional coat layer present in Active Particulates) containing, e.g., an 80:20, or higher, wt % ratio of nonionic polymer to pore former provides much better ODP compared to a functional coat with, e.g., a 60:40 wt % ratio of nonionic polymer to pore former, while maintaining a therapeutically acceptable immediate release of, e.g., an opioid(s) when taken in a manner consistent with manufacturer's instructions, or in a manner prescribed (e.g., one or two dosage units are taken as intended).

In certain embodiments, the pharmaceutically active agent is present in the dosage form in an amount effective for the intended therapeutic purpose. These amounts are well known in the art. Indeed, the doses at which any of the presently known active agents embraced by the present invention can be given safely and effectively for the intended therapeutic purpose are known to those of skill in the art. In certain embodiments, the active agent (e.g., an opioid) is present in an amount of about 0.1% to about 95% w/w of the Active Particulate before the addition of the (optional) seal coat, or any functional coat layer(s) (i.e., about 0.1% to about 95% w/w of the polymer matrix embedded with active agent). In certain embodiments, the active agent is present in an amount of about 0.2% to about 90%, about 0.3% to about 85%, about 0.4% to about 80%, about 0.5% to about 75%, about 0.6% to about 70%, about 0.7% to about 65%, about 0.8% to about 60%, about 0.9% to about 55%, about 1% to about 50%, about 2.5% to about 45%, about 5% to about 40%, about 7.5% to about 35%, about 10% to about 30%, about 12.5% to about 25%, or about 15% to about 20% w/w of the polymer matrix embedded with active agent. In certain embodiments, the active agent (e.g., opioid) is present in an amount of at least about 0.1%, at least about 0.2%, at least about 0.5%, at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% w/w of the polymer matrix embedded with active agent.

In certain embodiments, the active agents are drugs prone to abuse, misuse, and/or overdose. In certain embodiments, the active agents can include, without limitation, members of the therapeutic categories such as analgesics, anti-inflammatory agents, anthelmintics, anti-arrhythmic agents, anti-bacterial agents, anti-viral agents, anticoagulants, anti-depressants, anti-diabetic agents, anti-epileptic agents, anti-fungal agents, anti-gout agents, anti-hypertensive agents, anti-malarial agents, anti-migraine agents, anti-muscarinic agents, anti-neoplastic agents, erectile dysfunction improving agents, immunosuppressants, anti-protozoa agents, anti-thyroid agents, anti-anxiolytic agents, sedatives, hypnotics, neuroleptics, β-blockers, cardiac inotropic agents, corticosteroids, diuretics, anti-Parkinsonian agents, gastrointestinal agents, histamine receptor antagonists, keratolytics, lipid-regulating agents, anti-angina agents, cox-2 inhibitors, leukotriene inhibitors, macrolides, muscle relaxants, nutritional agents, protease inhibitors, sex hormones, stimulants, anti-osteoporosis agents, anti-obesity agents, cognition enhancers, anti-urinary incontinence agents, nutritional oils, anti-benign prostate hypertrophy agents, essential fatty acids, nonessential fatty acids, and any combinations of two or more thereof.

In certain embodiments, the active agent can be an opioid (e.g., an opioid analgesic). For example, without limitation, the opioid can be alfentanil, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, etorphine, dihydroetorphine, fentanyl, hydrocodone, hydromorphone, hydromorphodone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, nomiethadone, nalorphine, nalbuphene, normorphine, norpipanone, opium, oxycodone, oxymorphone, pantopon, papaveretum, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, tapentadol, tilidine, tramadol, pharmaceutically acceptable salts thereof.

In certain embodiments, the opioid can be oxycodone, hydrocodone, tapentadol, codeine, oxymorphone, hydromorphone, or pharmaceutically acceptable salts thereof. In certain embodiments, the opioid is oxycodone, hydrocodone, oxymorphone, hydromorphone, or codeine. In certain embodiments, the opioid is a pharmaceutically active salt of oxycodone, hydrocodone, oxymorphone, hydromorphone, or codeine.

In certain embodiments, the active agents can include, but are not limited to, benzodiazepines (e.g., bromazepam, chlordiazepoxied, clorazepate, diazepam, estazolam, flurazepam, halazepam, ketazolam, lorazepam, nitrazepam, oxazepam, prazepam, quazepam, temazepam, triazolam), barbiturates (e.g., amobarbital, aprobarbotal, butabarbital, butalbital, methohexital, mephobarbital, metharbital, pentobarbital, phenobarbital, secobarbital), and stimulants, such as amphetamines (e.g., amphetamine, dextroamphetamine resin complex, dextroamphetamine, methamphetamine, methylphenidate), as well as dronabinol, glutethimide, methylprylon, ethchlorovynol, ethinamate, fenfluramine, meprobamate, pemoline, levomethadyl, benzphetamine, chlorphentermine, diethylpropion, phentermine, mebutamate, chlortermine, phenylacetone, dronabinol, nabilone, chloral hydrate, ethclorovynol, paraldehyde, midazolam, and dextropropoxyphene, or pharmaceutically acceptable salts thereof.

Examples of pharmaceutically acceptable salt include, but are not limited to, citrate, oxalate, acetate, maleate, malonate, fumarate, succinate, tosylate, mesylate, hydrochloride, hydrobromide, sulfate, phosphate, methanesulfonate, toluenesulfonate or mixtures and/or forms thereof. Additional pharmaceutically acceptable salts can be found in P. H. Stahl and C. G. Wermuth, editors, Handbook of Pharmaceutical Salts: Properties, Selection and Use, Weinheim/Zirich:Wiley-VCH/VHCA, 2002.

### 5.2.2. Active Pellets

In certain embodiments, the Active Particulates are Active Pellets. In certain embodiments, the Active Pellets include an active agent and a functional coat layer(s). In certain embodiments, at least one of FC 0, FC 1, and FC 2 contain at least one cationic polymer and, optionally, a nonionic water-insoluble polymer. In certain embodiments, the Active Pellets can further include a seal coat (optional) between the polymer matrix (or alternate core) and a functional coat layer(s). In certain embodiments, the Active Pellets further include an over coat, comprising a water-soluble nonionic polymer, on top of the outermost functional coat layer(s). In certain embodiments, a functional coat, e.g., FC 1, includes a water-insoluble nonionic polymer, and a cationic polymer that is soluble in gastric fluids (e.g., at a pH less than about 5.0). The cationic polymer behaves as a pore former at a pH below about 5.0, but swells and becomes permeable at a pH above about 5.0 (e.g., in intestinal fluids), thereby substantially preventing release of the opioid at a higher pH.

In certain embodiments, the core of the Active Pellets can be preformed pellets. By way of example, but not limitation, the pellet core can be made from microcrystalline cellulose (MCC) and/or alkaline agents/ion exchange resins. In certain embodiments, the pellet core comprises MCC cellets containing cured PEO.

In certain embodiments, the shape of the pellets can be round, oval, or oblong.

In certain embodiments, that pellet core has a density of about 0.3 to about 1.0 mg/cm³.

In certain embodiments, the pellet core can be about 25 mg to about 500 mg. In certain embodiments, the pellet core can be about 50 mg to about 475 mg, about 75 mg to about 450 mg, about 100 mg to about 425 mg, about 125 mg to about 400 mg, about 150 mg to about 375 mg, about 175 mg to about 350 mg, about 200 mg to about 325 mg, about 225 mg to about 300 mg, or about 250 mg to about 275 mg.

In certain embodiments, the pellet core can be about 25% to about 90% w/w of the uncoated Active Pellet, i.e., the Active Pellet before being coated with an (optional) seal coat and/or a functional coat layer(s). In certain embodiments, the pellet core can be about 27.5% to about 87.5%, about 30% to about 85%, about 32.5% to about 82.5%, about 35% to about 80%, about 37.5% to about 77.5%, about 40% to about 75%, about 42.5% to about 72.5%, about 45% to about 70%, about 47.5% to about 67.5%, about 50% to about 65%, about 52.5% to about 62.5%, or about 55% to about 60% w/w of the uncoated Active Pellet.

In certain embodiments, Active Pellets (e.g., opioid-containing Opioid Pellets) contain an active agent (e.g., an opioid) in an amount of about 0.1% to about 95% w/w of the uncoated Active Pellets. In certain embodiments, e.g., Opioid Pellets contain the opioid in an amount of about 0.2% to about 90%, about 0.3% to about 85%, about 0.4% to about 80%, about 0.5% to about 75%, about 0.6% to about 70%, about 0.7% to about 65%, about 0.8% to about 60%, about 0.9% to about 55%, about 1% to about 50%, about 2.5% to about 45%, about 5% to about 40%, about 7.5% to about 35%, about 10% to about 30%, about 12.5% to about 25%, or about 15% to about 20% w/w of the uncoated Opioid Pellet. In certain embodiments, the Opioid Pellets contain the opioid in an amount of at least about 0.1%, at least about 0.2%, at least about 0.3%, at least about 0.4%, at least about 0.5%, at least about 0.75%, at least about 1%, at least about 2.5%, at least about 5%, at least about 7.5%, at least about 10%, at least about 12.5%, at least about 15%, at least about 17.5%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% w/w of the uncoated Opioid Pellet.

In certain embodiments, the opioid is oxycodone, or a pharmaceutically acceptable salt thereof. In certain embodiments, the opioid is oxycodone hydrochloride. In certain embodiments, the opioid is hydrocodone, or a pharmaceutically acceptable salt thereof. In certain embodiments, the opioid is hydrocodone bitartrate. In certain embodiments, the opioid is hydromorphone, or a pharmaceutically acceptable salt thereof. In certain embodiments, the opioid is hydromorphone hydrochloride. In certain embodiments, the opioid is oxymorphone. In certain embodiments, the opioid is codeine, or a pharmaceutically acceptable salt thereof.

In certain embodiments, the active agent can be absorbed by the pellet core. In certain embodiments, the active agent can be coated onto the pellet core. In certain embodiments, the active agent can be dissolved into a suitable solvent system to either be absorbed by the pellet core or sprayed onto the pellet core. In certain embodiments, the solvent is water, an alcohol, an organic liquid, or a combination thereof. In certain embodiments, the alcohol is a dehydrated alcohol. In certain embodiments, the solvent is a mixture of water and an alcohol. In certain embodiments, the solvent is a mixture of water and a dehydrated alcohol. In certain embodiments, the components of a solvent mixture can be added at the same time or in different steps or stages.

In certain embodiments, solvents that can be used in processes of preparing dosage forms of the present disclosure (e.g., dosage forms comprising Active Pellets) include, but are not limited to, water, methanol, ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulfoxide, N,Ndimethylformamide, tetrahydrofuran, and any mixtures thereof.

In certain embodiments, the active agent coating may also contain additives such as coloring agents, talc and/or magnesium stearate, which are well known in the coating arts. In certain embodiments, the excipients added to the active agent solution can include, but are not limited to hydroxypropylmethylcellulose (HPMC) (e.g., methocel E5 Premium LV), lactose, polyvinylpyrrolidone (PVP), magnesium stearate, and talc. In certain embodiments, the excipients can be present in an amount of about 0.1% to about 30% w/w of the uncoated Active Pellet. In certain embodiments, the Active Pellets contain excipients in an amount of about 0.2% to about 27.5%, about 0.3% to about 25%, about 0.4% to about 22.5%, about 0.5% to about 20%, about 0.6% to about 17.5%, about 0.7% to about 15%, about 0.8% to about 12.5%, about 0.9% to about 10%, about 1% to about 7.5%, or about 2.5% to about 5% w/w of the uncoated Active Pellet. In certain embodiments, the Active Pellets contain excipients in an amount of at least about 0.1%, at least about 0.2%, at least about 0.5%, at least about 1%, at least about 5%, at least about 10%, at least about 15%, at least about 20%, at least about 25%, or at least about 30% w/w of the uncoated Active Pellet.

In certain embodiments, Active Pellets can be made by coating the active agent upon the pellet core. For example, Active Pellets can be made by the following steps:
1. Add oxycodone hydrochloride to a solvent system containing at least one component (e.g., dehydrated alcohol) taken in a suitable size stainless steel container and mix until it disperses uniformly.
2. While mixing, gradually add excipients (e.g., HPMC, talc) until it disperses uniformly.
3. Add purified water to the dispersion from step #2 and mix until a clear solution is formed.
4. Coat the pellets using a fluid bed coater with an inlet air temperature of 40°-50° C. and sufficient air volume for fluidization.
5. When the product temperature reaches 30° C., start spraying the dispersion from step #4 onto pellets while maintaining the product temperature of 28-30° C. and sufficient air volume for the fluidization until the target coating weight gain is reached.
6. Dry the coated pellets from step #5.

### 5.2.3. Active Granules

In certain embodiments, the Active Particulates are Active Granules. In certain embodiments, the Active Granules include an active agent, a polymer matrix that in some embodiments may include hydrophilic polyoxyethylene (PEO) polymer, a cationic polymer or a nonionic polymer, an antioxidant, a plasticizer and a surfactant. In certain embodiments, the Active Granules may include a seal coat and at least one functional coat layer(s) (e.g., FC 1). In certain embodiments, the seal coat is optional. In certain embodiments, Active Granules containing, e.g., FC 1 can further include FC 0 between the polymer matrix and FC 1. In certain embodiments, the Active Particulates include FC 2 over FC 1. In certain embodiments, the Active Particulates include an over coat, comprising a water-soluble nonionic polymer, surrounding the outermost functional coat layer(s). In certain embodiments, at least one of FC 0, FC 1, and FC 2 includes a water-insoluble nonionic polymer (e.g., generally not soluble in physiological fluids and commonly used organic solvents such as ethanol) and a cationic polymer. The latter behaves as a pore former at a pH below about 5.0, but swells and becomes partially permeable at a pH above 5.0 (e.g., in intestinal fluids, or in gastric fluids with an elevated pH), thereby substantially preventing release of the active agent (e.g., an opioid) at higher pH.

In certain embodiments, Active Granules may contain a plasticizer in the polymer matrix, the outer coatings (e.g., the seal coat, the functional coat layer(s), and/or the over coat), or both the polymer matrix and the outer coatings. In certain embodiments, the Active Granules may contain a surfactant in the polymer matrix, the outer coatings, or in both the polymer matrix and the outer coatings.

In certain embodiments, Active Granules contain an active agent (e.g., an opioid) in an amount of about 0.1% to about 95% w/w of the uncoated Active Granules, i.e., the Active Granules before being coated with the (optional) seal coat and/or any functional coat layer(s).

In certain embodiments, the active agent is an opioid. In certain embodiments, the opioid is oxycodone, or a pharmaceutically acceptable salt thereof. In certain embodiments, the opioid is oxycodone hydrochloride. In certain embodiments, the opioid is hydrocodone, or a pharmaceutically acceptable salt thereof. In certain embodiments, the opioid is hydrocodone bitartrate. In certain embodiments, the opioid is hydromorphone, or a pharmaceutically acceptable salt thereof. In certain embodiments, the opioid is hydromorphone hydrochloride. In certain embodiments, the opioid is oxymorphone. In certain embodiments, the opioid is codeine, or a pharmaceutically acceptable salt thereof.

In certain embodiments, the polymer matrix comprises a nonionic polymer and/or a cationic polymer. Representative cationic polymers include, but are not limited to, (meth)acrylic polymers and (meth)acrylic copolymers (e.g., copolymers of alkyl (meth)acrylates and copolymers of alkylamino(meth)acrylates); quarternary ammonium (meth)acrylic polymers.

Representative nonionic polymers include, but are not limited to, a nonionic copolymer of ethyl acrylate, methyl methacrylate and a low content of methacrylic acid ester with quaternary ammonium groups (ammonium methacrylate copolymer, Type A, NF) (e.g., EUDRAGIT® RL 100, RS100 (Evonik)); and nonionic polymers such as hydroxypropylcellulose (e.g., KLUCELE®, L, J, G, M and H grades (Ashland)), hydroxypropyl methylcellulose (HPMC) (e.g., METHOCEL® E, F, J, and K (Dow Chemicals)), hydroxyethylcellulose (e.g., NATRASOL L, G, M, and H grades (Ashland)), ethylcellulose (e.g., ETHOCEL® 7FP, 10FP, 45FP, and 100FP (Dow Chemicals) and N7, N10, N14, N22, N50, and N100 grades (Ashland)), cellulose acetate butyrate (e.g., CAB-381-0.5 (Eastman)), and cellulose acetate (CA-398-3, CA-398-6, CA-398-100, and CA-398-30 (Eastman)); polyvinyl acetate polymers (e.g., polyvinyl acetate-polyvinylpyrrolidone (Kollidon SR) and polyethylene oxide polymers (e.g., Polyox® WSR coagulant, Polyox® WSR-301, Polyox® WSR-303). Exemplary polyoxyethylene oxide polymers include POLYOX™ WSR N-80, POLYOX™ WSR N-750, POLYOX™ WSR N-3000, POLYOX™ WSR-205, POLYOX™ WSR N-1105, POLYOX™ WSR N-12K, POLYOX™ WSR N-60K, POLYOX™ WSR N-301, POLYOX™ WSR Coagulant, POLYOX™ WSR N-303. The exemplary polyoxyethylene oxide polymers provide different viscosities in an aqueous solution. In certain embodiments, the exemplary polyethylene oxide has an average molecular weight of about 1,000,000 (WSR-N-12K), about 4,000,000 (WSR-301), about 5,000,000 (WSR Coagulant), or about 7,000,000 (WSR-303).

Representative pH-dependent polymers include, but are not limited to, cationic pH-dependent release polymers that are soluble in gastric fluid, but swell and become permeable at a pH above 5.0. In some embodiments, the cationic pH-dependent polymer matrix comprises EUDRAGIT® E PO which has a molecular weight about 47,000 and a glass transition temperature about 48° C.

The polymer matrix (i.e., the polymer matrix without the active agent embedded within) may be present in the Active Granules in a range of about 1.0% to about 95% w/w based on the total weight of the uncoated Active Granule, in some embodiments, from about 15% to about 90% w/w based on the total weight of the uncoated Active Granule, and in other embodiments, from about 30% to about 75% w/w based on the total weight of the uncoated Active Granule. In certain embodiments, the polymer matrix may be present in an amount of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 95% w/w based on the total weight of the uncoated Active Granule.

In certain embodiments, a plasticizer may be added to increase the elasticity of the polymer in Active Granules. In certain embodiments, the plasticizer makes the Active Granule crush-resistant. In certain embodiments, the plasticizer is soluble in both aqueous and nonaqueous solvents that are commonly used to extract opioids and other abuse-prone drugs from commercial formulations. In certain embodiments, the plasticizer acts as an aversion agent. In certain embodiments, the plasticizer acts as a tissue irritant that causes discomfort if administered in conjunction with an active agent with which it is coextracted.

Representative plasticizers include, but are not limited to liquid esters, (e.g., triethyl citrate, propylene glycol, polyethylene glycols, triacetin, diethylene glycol monoethyl ether, dibutyl sebacate, and diethyl phthalate). In certain embodiments, the dielectric constant values of the plasticizer are in a range of about 5 to about 60. In other embodiments, the dielectric constant values of the plasticizer are in a range of about 10 to about 40.

In certain embodiments, the plasticizer may be present in an amount that is sufficient to make the Active Granules substantially crush-resistant, but not in quantities that negatively impact the dissolution of the active agent when taken in a manner consistent with the manufacturer's instructions or in a manner not prescribed. In certain embodiments, the plasticizer may be present in amounts that result in discomfort to the abuser when the plasticizer is co-eluted with the active agent and administered in a manner inconsistent with the manufacturers and/or physicians instructions. In certain embodiments, the amount of plasticizer provides an adequate rubbery state and elongation property to the polymer to achieve crush-resistance, making it difficult to pulverize the Active Granules into a fine powder, thereby deterring abuse.

In certain embodiments, the plasticizer may be present in a range of about 0.1% to about 30% w/w of the uncoated Active Granules. In certain embodiments, the plasticizer may be present in a range from about 2.0% to about 15% w/w of the uncoated Active Granules. In certain embodiments, the plasticizer may be present in an amount of about 0.2% to about 27.5%, about 0.3% to about 25%, about 0.4% to about 22.5%, about 0.5% to about 20%, about 0.6% to about 17.5%, about 0.7% to about 15%, about 0.8% to about 12.5%, about 0.9% to about 10%, about 1% to about 7.5%, or about 2.5% to about 5% w/w of the uncoated Active Granule. In certain embodiments, the plasticizer may be present in an amount of at least about 0.1%, at least about 0.2%, at least about 0.5%, at least about 1%, at least about 5%, at least about 10%, at least about 15%, at least about 20%, at least about 25%, or at least about 30% w/w of the uncoated Active Granule. In certain embodiments, the plasticizer may be present in an amount of about 2%, about 3%, about 4%, about 6%, or about 8% w/w of the uncoated Active Granule.

In certain embodiments, the Active Granule matrix further comprises at least one surfactant. In certain embodiments, the pharmaceutically acceptable surfactants that are useful in the practice of the present invention have solubility in oils, co-solvents, or aqueous media. In certain embodiments, the surfactant component helps in modulating the solubility of the active agent. In certain embodiments, the surfactant helps to reducing the abuse potential by a dual mechanism. First, it elicits the irritant response when administered “as is” by nasal or injection routes, and second, by co-eluting with the drug when extracted with the commonly used solvents such as aqueous and organic solvents. Surfactants produce tissue irritation when applied to nasal mucosa and will cause local irritation at an injection site. Further, docusate sodium is commonly used as a stool softener/laxative, so while providing some relief for opioid-induced constipation at the intended dose, it can cause undesirable gastrointestinal effects if large quantities are ingested. Similar gastrointestinal effects can be obtained by ingesting other surfactants. In certain embodiments, the surfactant is present in an amount that results in discomfort to the abuser when the surfactant is co-eluted with the pharmaceutically active agent. The hydrophilic-lipophilic balance (“HLB”) values of the surfactants are in a range of about 4 to about 30.

Types of surfactants that may be useful in the practice of the present invention include nonionic surfactants (e.g., esters of fatty acids, especially of C8-C24 and preferably of C16-C22, and fatty acid esters of polyols such as glycerol or sorbitol); sorbitan fatty acid esters ethoxylated with from 2 to 30 moles of ethylene oxide; polyethylene glycol fatty acid esters; polyethyleneglycol esters and polyethyleneglycol ethers; and polyethoxylated carboxylic acids (e.g., PEG-35 castor oil, PEG-40 castor oil, steareth-2 (e.g., Brij 72, Uniqema), steareth-21 (e.g., Brij 721, Uniqema), ceteareth-25 (e.g., Cremophor A25, BASF Cooperation), PEG-7 hydrogenated castor oil (e.g., Cremophor WO7, BASF Cooperation), and PEG-30 Dipolyhydroxystearate (e.g., Arlacel P 135, Uniqema)); block copolymers based on ethylene oxide and propylene oxide (e.g., PLURONIC® (e.g., 188 or 407 (BASF)); dioctyl sodium sulfosuccinate (docusate sodium); sodium lauryl sulfate; PEG-32 glyceryl laurate; PEG-32 glyceryl palmitostearate; PEG-8 glyceryl caprylate/caprate; PEG-6 glyceryl caprylate/caprate; macrogol 15 hydroxystearate; polyoxyethylene 20 sorbitan monolaurate (polysorbate 20); polyoxyethylene 20 sorbitan monooleate (polysorbate 80); sorbitan monolaurate; sorbitan monooleate; and polyoxyl 40 stearate. Anionic surfactants (e.g., alkyl ether sulfates and sulfosuccinates) may also be useful. Alternatively cationic and amphoteric surfactants such as phospholipids, lysophospholipids, and pegylated phospholipids may also be used. Additional useful surfactants include, vitamin E and derivatives thereof (e.g., PEGylated derivatives of vitamin E such as tocopherol PEG succinate, tocopheryl polyethylene glycol sebacate, tocopheryl polyethylene glycol dodecanodioate, tocopheryl polyethylene glycol suberate, tocopheryl polyethylene glycol azelaate, tocopheryl polyethylene glycol citraconate, tocopheryl polyethylene glycol methylcitraconate, tocopheryl polyethylene glycol itaconate, tocopheryl polyethylene glycol maleate, tocopheryl polyethylene glycol glutarate, tocopheryl polyethylene glycol glutaconate, tocopheryl polyethylene glycol fumarate, tocopheryl polyethylene glycol phthalate, tocotrienol polyethylene glycol succinate, tocotrienol polyethylene glycol sebacate, tocotrienol polyethylene glycol dodecanodioate, tocotrienol polyethylene glycol suberate, tocotrienol polyethylene glycol azelaate, tocotrienol polyethylene glycol citraconate, tocotrienol polyethylene glycol methylcitraconate, tocotrienol polyethylene glycol itaconate, tocotrienol polyethylene glycol maleate, tocotrienol polyethylene glycol glutarate, tocotrienol polyethylene glycol glutaconate, tocotrienol polyethylene glycol fumarate, and tocotrienol polyethylene glycol phthalate). See, e.g., USPAP 2014/0271593, hereby incorporated-by-reference herein.

In certain embodiments, the surfactant may be present in a range of about 0.01% to about 15% w/w of the uncoated Active Granules. In certain embodiments, the surfactant may be present in a range from about 0.15% to about 5% w/w of the uncoated Active Granules. In certain embodiments, the surfactant may be present in an amount of about 0.025 to about 12.5%, about 0.05% to about 10%, about 0.075% to about 7.5%, about 0.1% to about 5%, about 0.25% to about 2.5%, or about 0.5% to about 1% w/w of the uncoated Active Granules. In certain embodiments, the surfactant may be present in an amount of about 0.2%, about 0.5%, about 2%, or about 2.2%, w/w of the uncoated Active Granules.

In certain embodiments, certain combinations of aversion agents (e.g., plasticizer and surfactant) can be used to deter abuse. Examples of such combinations include, but are not limited to, triethyl citrate and docusate sodium (DOSS™); propylene glycol and DOSS™; polyethylene glycol (PEG-400) and DOSS™; and PEG-400 or PEG-40 hydrogenated castor oil. In certain embodiments, surfactants are used as aversion agents. Examples of such surfactants include, but are not limited to, Polyoxyl 40 hydrogenated castor oil (Cremaphor RH40), PEG 35 castor oil, and Polyoxyl 35 hydrogenated castor oil (Cremaphor EL). In certain embodiments, plasticizers are used as aversion agents. Examples of such plasticizers include, but are not limited to, PEG-3350 and PEG-6000.

In certain embodiments, the Active Granules further contain an antioxidant. In certain embodiments, the antioxidants are present in an amount sufficient to suppress degradation of high molecular weight PEO upon hot melt extrusion (HME). Polymer degradation may result in an uncontrolled release profile, particularly when active material is embedded in a matrix of PEO; this may be another cause of oxidative degradation of pharmacologically active ingredients by, e.g., radicals. When adding an excipient, such as butylated hydroxytoluene (BHT), in order to attempt to stabilize high molecular weight PEO polymer, it should be taken into consideration that such an excipient should be stable at elevated temperatures, e.g., hot-melt extrusion temperatures used during manufacture of Active Granules. Antioxidants for use in the present invention include, but are not limited to, ascorbic acid and its salts, tocopherols, sulfite salts such as sodium metabisulfite or sodium sulfite, sodium sulfide, butylated hydroxyanisole, butylated hydroxytoluene, ascorbyl palmitate, and propyl gallate. In certain embodiments, the antioxidant may be present in a range of about 0.01% to about 2% w/w of the uncoated Active Granules. In certain embodiments, the antioxidant may be present in a range of about 0.025% to about 1%, about 0.05% to about 0.75%, about 0.075% to about 0.5%, or about 0.1 to about 0.75% w/w of the uncoated Active Granules. In certain embodiments, the antioxidant may be present in about 0.2%, about 0.3%, about 0.4%, or about 0.5% w/w of the uncoated Active Granules.

In certain embodiments, the Active Granules may be prepared in several ways known to those in the art, including HME, film melt, granulation, melt granulation, extrusion spheronization, or rotor or roller compaction. In certain embodiments, the Active Granules, containing PEO polymers, prepared by granulation, extrusion (e.g., HME), spheronization, rotor, or roller compaction process may require curing at a temperature above the melting point of the PEO polymers. In certain embodiments, the Opioid Granules may be prepared by an HME process. In an HME process, a thermoplastic carrier polymer (e.g., nonionic polymer and/or cationic polymer) is combined with an active agent, a plasticizer, a surfactant, as well as any optional ingredients (e.g., an ion exchange polymer, alkaline buffering agent, and/or viscosity-building agent) to form a powdery mixture. The mixture is introduced into one or two rotating screws that convey the powder into a heated zone where shear forces compound the materials until a molten mass is achieved. Hot-melt extrusion equipment typically includes an extruder, auxiliary equipment for the extruder, downstream processing equipment, and other monitoring tools used for performance and product quality evaluation. The extruder is typically composed of a feeding hopper, barrels, single or twin screws, and the die and screw-driving unit. The auxiliary equipment for the extruder mainly includes a heating/cooling device for the barrels, a conveyer belt to cool down the product, and a solvent delivery pump. The monitoring devices on the equipment include temperature gauges, a screw-speed controller, an extrusion torque monitor and pressure gauges. In certain embodiments, different shaped dies can be used. For example, extrudates can be produced by extruding the material through round-shaped dies into cooled rolls, wherein the extruded strands are cut into short cylinders using a pelletizer.

The pelletized extruded strands are subjected to an appropriate size reduction process(es) using co-mill or fitz mill or micropulverizer with coolant processing aids such as dry ice or liquid nitrogen.

In certain embodiments, the sizes of Active Granules, before or after attempted grinding, are significantly large enough to prevent the granules from being snorted. In certain embodiments, the mean size distribution of the Active Granules can be from about 125 μm to about 1000 μm (1 mm), and in some embodiments from about 250 μm to about 750 μm (as measured by weight frequency distribution using sieving method). In certain embodiments, the mean particle size of the Active Granules is about 400 μm to about 600 μm. In certain embodiments, the mean particle size of the Active Granules is about 500 μm.

### 5.2.4. Seal Coat

In certain embodiments, the Active Particulates may be seal coated. The seal coat may be disposed between the inner polymer matrix core (i.e., the polymer matrix with active agent embedded within) and the at least one functional coat (i.e., FC 1). In certain embodiments, the seal coat can be made with a nonionic water-soluble polymer. In certain embodiments, the nonionic water soluble polymer that can be included in the seal coat is a cellulose ether polymer (e.g., a water-soluble methylcellulose and/or hydroxypropylmethylcellulose polymer). In certain embodiments, the amount of the polymer ranges from about 5% to about 100% w/w of the total weight of the composition of the seal coat (also noted within as “seal coat composition”), in some embodiments from about 30% to about 95% w/w based on the total weight of the composition of the seal coat and in some embodiments from about 50% to about 75% w/w based on the total weight of the seal coat composition. In certain embodiments, the amount of the polymer ranges from about 10% to about 95%, about 15% to about 90%, about 20% to about 85%, about 25% to about 80%, about 30% to about 75%, about 35% to about 70%, about 40% to about 65%, about 45% to about 60%, or about 50% to about 55% w/w of the total weight of the seal coat composition.

In certain embodiments, the composition of the seal coat may also include additional excipients such as an anti-tacking agent (e.g., talc, magnesium trisilicate, colloidal silicon dioxide (e.g., CAB-O-SIL®)) and a plasticizer; the plasticizer may be the same as or different from the plasticizer(s) that may be present in Active Particulates. In certain embodiments, the amount of the additional excipients, when present, can range from about 0.1% to about 40% w/w of the total weight of the seal coat composition, and in some embodiments from about 0.5% to about 10% w/w based on the total weight of the seal coat composition. In certain embodiments, the additional excipients are present at about 0.5% or about 4% w/w based on the total weight of the seal coat composition. In certain embodiments, the additional excipients are present at about 0.25% or about 35%, about 0.5% or about 30%, about 0.75% or about 25%, about 1% or about 20%, about 2.5% or about 15%, or about 5% or about 10% w/w based on the total weight of the seal coat composition.

In certain embodiments, the seal coat composition may also include an amount of the active agent, which may be therapeutically effective in and of itself, as well as the plasticizer and/or the surfactant, as well as other excipients and ingredients such as one or more solvents (both aqueous and organic, e.g., ethanol), as well as other excipients that may also be included in the seal coat composition.

In certain embodiments, the seal coat may be present in a range of about 0.1% to about 40% w/w of the uncoated Active Particulates, i.e., the Active Particulates before being coated with the (optional) seal coat, the Functional Coat(s), and the over coat. In certain embodiments, the seal coat may be present in a range from about 5% to about 25% w/w of the uncoated Active Particulates. In certain embodiments, the seal coat may be present in an amount of about 5% or about 15% w/w of the uncoated Active Particulates. In certain embodiments, the seal coat may be present in a range of about 0.2% to about 37.5%, about 0.3% to about 35%, about 0.4% to about 32.5%, about 0.5% to about 30%, about 0.6% to about 27.5%, about 0.7% to about 25%, about 0.8% to about 22.5%, about 0.9% to about 20%, about 1% to about 17.5%, about 2.5% to about 15%, about 5% to about 12.5%, or about 7.5% to about 10% w/w of the total weight of the uncoated Active Particulates. In certain embodiments, the seal coat may be present in an amount of at least about 0.1%, at least about 0.2%, at least about 0.5%, at least about 1%, at least about 5%, at least about 10%, at least about 15%, at least about 20%, at least about 25%, at least about 30%, at least about 35%, or at least about 40% w/w of uncoated Active Particulates.

### 5.2.5. Functional Coat Layers

In certain embodiments, the Active Particulates are coated with a functional coat layer(s) (e.g., FC 0, FC 1, and/or FC 2). In certain embodiments, one or more functional coat layers, e.g., FC 1, include a water insoluble nonionic polymer (such as a polymer that is not soluble in physiological fluids and common organic solvents such as ethanol) and a cationic polymer (such as a polymer that is soluble in gastric fluids) that behaves as a pore former at pH below about 5.0.

In certain embodiments, functional coat layer(s) of the Active Particulates may comprise at least a water-insoluble nonionic polymer, e.g., cellulose acetate, cellulose acetate-based polymers (e.g. OPADRY® CA, cellulose acetate butyrate, cellulose acetate propionate, and the like), polyvinyl acetate polymers, polyvinyl acetate-based copolymers (e.g., KOLLIDON® SR), ethylcellulose (e.g., ETHOCEL™), EUDRAGIT® RL 100, EUDRAGIT® RL PO, EUDRAGIT® RS 100, EUDRAGIT® RS PO, EUDRAGIT® NE 30 D, EUDRAGIT® NE 40 D, and the like, or a blend thereof; and a pH-dependent, cationic copolymer (e.g., dimethylaminoethyl methacrylate, butyl methacrylate, and methyl methacrylate copolymer (e.g., EUDRAGIT® E PO)).

In certain embodiments, the functional coat layer(s) comprises at least cellulose acetate and a dimethylaminoethyl methacrylate, butyl methacrylate, and methyl methacrylate copolymer. In certain embodiments, the dimethylaminoethyl methacrylate, butyl methacrylate, and methyl methacrylate copolymer is EUDRAGIT® E PO.

In certain embodiments, cellulose acetate (“CA”) and/or CA-based polymer blends, together with the pH-dependent pore former, becomes almost impermeable at a pH greater than about 5.0, thereby significantly reducing drug release. In certain embodiments, the ratio of CA to pore former (i.e., CA: pore former) can be from about 50:50 to about 98:2 wt % ratio, or from about 70:30 to about 98:2 wt % ratio. In certain embodiments, the ratio of CA to pore former can be from about 72.5:27.5 to about 95:5, about 75:25 to about 92.5:7.5, about 77.5:22.5 to about 90:10, about 80:20 to about 87.5:12.5, or about 82.5:17.5 to about 85:15 wt % ratio. In certain embodiments, the ratio of CA to pore former can be about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7 about 94:6 about 95:5, about 96:4, about 97:3, or about 98:2 wt % ratio. In certain embodiments, the ratio of CA to pore former can be about 80:20 wt % ratio.

In certain embodiments, the nonionic water-insoluble polymer is a polyvinyl acetate polymer (“PVA polymer”) or a PVA-based polymer or copolymer. In certain embodiments, the PVA-based polymer along with the pH-dependent pore former becomes almost impermeable at pH greater than 5.0, thereby significantly reducing drug release. In certain embodiments, the ratio of PVA-based polymer to pore former (i.e., PVA-based polymer: pore former) can be from about 70:30 to about 98:2 wt % ratio. In certain embodiments, the ratio of PVA-based polymer to pore former can be from about 72.5:27.5 to about 95:5, about 75:25 to about 92.5:7.5, about 77.5:22.5 to about 90:10, about 80:20 to about 87.5:12.5, or about 82.5:17.5 to about 85:15 wt % ratio. In certain embodiments, the ratio of PVA-based polymer to pore former can be about 71:29, about 72:28, about 73:27, about 74:26, about 75:25, about 76:24, about 77:23, about 78:22, about 79:21, about 80:20, about 81:19, about 82:18, about 83:17, about 84:16, about 85:15, about 86:14, about 87:13, about 88:12, about 89:11, about 90:10, about 91:9, about 92:8, about 93:7 about 94:6 about 95:5, about 96:4, about 97:3, or about 98:2 wt % ratio. In certain embodiments, the ratio of PVA-based polymer to pore former can be about 80:20 wt % ratio.

In certain embodiments, if three or more dosage units are taken, release of the active agent from the dosage form is significantly reduced. In certain embodiments, the release is reduced by 25%, 35%, 45%, 55%, 65%, 75%, 85%, 95%, 96%, 97%, 98%, 99%, or increments therein. In certain embodiments, the release is reduced from about 30% to about 90%, about 40% to about 80%, or about 50% to about 70%.

In certain embodiments, the composition of the functional coating may also include an anti-tacking agent (e.g., talc, magnesium trisilicate, colloidal silicon dioxide (e.g., CAB-O-SIL®)) and/or a plasticizer.

In certain embodiments, the functional coating prevents the extraction of the active agent in water and in water/alcohol mixtures.

In certain embodiments, FC 1 may be present in a range of about 5% to about 70% w/w of the uncoated or seal coated Active Particulates (e.g., the polymer matrix with active agent embedded within, also including the optional seal coat, if present). In certain embodiments, the FC 1 may be present in a range of about 10% to about 65%, about 15% to about 60%, about 20% to about 55%, about 25% to about 50%, about 30% to about 45%, or about 35% to about 40% w/w of the uncoated or seal coated Active Particulates. In certain embodiments, FC 1 may be present in a range of about 5% to about 10%, about 5.25% to about 9.75%, about 5.5% to about 9.5%, about 5.75% to about 9.25%, about 6% to about 9%, about 6.25% to about 8.75%, about 6.5% to about 8.5%, or about 6.75% to about 8.25% w/w of the uncoated or seal coated Active Particulates. In certain embodiments, FC 1 may be present in a range from about 10% to about 35%, or about 15% to about 25% w/w of the uncoated or seal coated Active Particulates.

In certain embodiments, the functional coated Active Particulates may be further coated with an additional functional coat layer(s) (e.g., FC 2 and/or FC 0) to further enhance ODP features. In certain embodiments, FC 2 and/or FC 0 can comprise a cationic polymer (e.g., EUDRAGIT® E PO). In certain embodiments, FC 2 and/or FC 0 can comprise a cationic polymer and a nonionic polymer.

In certain embodiments, the composition of the FC 2 and/or FC 0 can also include an anti-tacking agent (e.g., talc, magnesium trisilicate, colloidal silicon dioxide (e.g., CAB-O-SIL)) and/or a plasticizer.

In certain embodiments, Active Particulates can comprise one, two, or three functional coat layer(s) (e.g., FC 1, or FC 1 and FC 0 and/or FC 2). In certain embodiments, Active Particulates can comprise more than three functional coat layers (e.g., four or five functional coat layers). In certain embodiments, any one or more of the functional coat layers can comprise a cationic polymer(s) in the absence of a water-insoluble nonionic polymer. In certain embodiments, any one or more of the functional coats can comprise a cationic polymer(s) in the presence of a water-insoluble nonionic polymer; in such embodiments, the ratio of nonionic polymer to cationic polymer can be from about 0.1:99.9 to about 99.9:0.1.

### 5.2.6. Over Coat

In certain embodiments, the functional coated Active Particulates (i.e., with or without FC 2) include an over coat to prevent/minimize the interaction of EUDRAGIT® E PO (e.g., in FC 1 and/or FC 2) with the alkaline agent present in the Triggering Particulates. The over coat may include a nonionic polymer (e.g., hydroxypropyl methylcellulose).

In certain embodiments, the composition of the over coat may also include additional excipients such as an anti-tacking agent (e.g., talc, magnesium trisilicate, colloidal silicon dioxide (e.g., CAB-O-SIL®)) and a plasticizer; the plasticizer may be the same as or different from the plasticizer(s) that may be present in Active Particulates.

In certain embodiments, the over coat may be present in a range of about 5% to about 50% w/w of the functional coated Active Particulates (i.e., the polymer matrix with active agent embedded within, (optional) seal coat, and one or more functional coat layers). In certain embodiments, the over coat may be present in a range of about 10% to about 50%, about 10% to about 45%, about 10% to about 35%, about 10% to about 30%, about 15% to about 40%, about 15% to about 25%, about 20% to about 35%, or about 25% to about 30% w/w of the functional coated Active Particulates.

### 5.2.7. Crush and Extractability Resistance

In certain embodiments, the Active Granules are at least partially crush-resistant, nongrindable, and nonextractable. In certain embodiments, they are substantially noncrushable, nongrindable, and nonextractable, thereby making the active agent difficult to abuse. For example, the Active Granules resist abuse via, but not limited to, crushing and swallowing; crushing and insufflating/inhaling nasally (“snorting”); crushing and smoking; or crushing, dissolving, and injecting (subcutaneously (i.e., skin popping), intravenously, or intramuscularly). In certain embodiments, the Active Granules cannot be ground or crushed into particles small enough to be effectively snorted or injected. In certain embodiments, the Active Granules cannot be pulverized into fine powder by mechanical grinding.

The crush-resistance of the Active Granules may be determined by a measurement of crushing strength required to deform the granules without any evidence of fragmentation, or breaking into smaller pieces or powder using an Instron Tester or equivalent. In some embodiments, the active granules may withstand a crushing strength ranging from 300-1000 N. Abuse deterrence can be tested by examining the mean particle size following the physical and/or mechanical manipulation, with or without thermal pretreatment, of the Active Granule. For example, the Active Granules can be subjected to grinding/crushing in a coffee grinder, mill, mortar and pestle, a food processor, a blender, etc. For example, Active Granules can be placed in a coffee grinder (e.g., Hamilton Beach Coffee Grinder) and ground for several cycles (e.g., at a 10 cup setting for 8 cycles of 30 seconds each).

The mean particle size of the granules after grinding can be measured using sieve analysis that gathers granules of the same size into groups based on particle size. The weight of the particles in each group can be measured and compared to an unground sample.

In certain embodiments, the mean particle size after grinding the Active Granules is about 500 μm (with a range of about 250 μm to about 1000 μm), as measured by weight frequency distribution using sieving method. In certain embodiments, the mean particle size after grinding the Active Granules is greater than about 150 μm, about 175 μm, about 200 μm, about 225 μm, about 250 μm, about 275 μm, about 300 μm, about 325 μm, about 350 μm, about 375 μm, about 400 μm, about 425 μm, about 450 μm, about 475 μm, about 500 μm, about 525 μm, about 550 μm, about 575 μm, about 600 μm, about 625 μm, about 650 μm, about 675 μm, or about 700 μm.

Abuse deterrence can be tested by examining the syringeability of the Active Granules either before or after grinding. For example, syringeability can be tested by examining the difficulty of drawing a solution of the dosage form, dissolved in varying types of solvents (e.g., water) and volumes of solvent (e.g., 2-10 ml) through, e.g., an 18 gauge syringe needle. The syringeability can also be tested by determining the amount of active ingredient present in the withdrawn liquid.

Abuse deterrence can also be tested by examining the extractability of active agent from the Active Granules before and after grinding.

### 5.3. Triggering Particulates

In certain embodiments, the Triggering Particulates can be Triggering Granules. In certain embodiments, the Triggering Granules can contain a combination of at least one alkaline agent (e.g., magnesium hydroxide (increases pH from 1.6 to greater than 5.0)) and/or at least one pH-stabilizing agent (e.g., di- and/or tricalcium phosphate (maintains the elevated pH of greater than 5.0 for up to about 30 minutes, about one hour, or about two hours)). In certain embodiments, ingestion of one dosage unit (i.e., one tablet or capsule) results in little or no increase in pH of the gastric fluids. In certain embodiments, ingestion of multiple dosage units (e.g., three or more) results in the alkaline agent increasing the pH very rapidly above about 5.0. In certain embodiments, the pH-stabilizing agent acts to maintain or stabilize the increased pH caused by the alkaline agent. For example, ingestion of multiple dosage units results in (a) a rapid increase in pH caused by the alkaline agent; (b) modulation of pore formation in the functional coat; and (c) a decrease in the rate of release of the active agent (e.g., an opioid) from the Active Particulate. In certain embodiments, upon ingestion of multiple dosage units (e.g., three or more), the pH of the gastric fluid increases very rapidly above a pH of about 5.0 (e.g., in about one to about five minutes). In certain embodiments, the increase in the pH of the gastric fluid upon taking multiple dosage units occurs in about two to about three minutes.

In certain embodiments, the alkaline agent for use in the Triggering Granules include, but are not limited to, aluminum hydroxide, sodium hydroxide, potassium hydroxide, calcium hydroxide, magnesium hydroxide, calcium carbonate, sodium carbonate, potassium bicarbonate, sodium bicarbonate, sodium oxide, calcium oxide, magnesium oxide, aluminum oxide, potassium oxide, ammonia, tertiary sodium phosphate, diethanolamine, ethylenediamine, N-methylglucamine, L-lysine, and combinations thereof. In certain embodiments, the alkaline agent is magnesium hydroxide.

In certain embodiments, the alkaline agent is present in an amount that when a single dosage unit is taken, it does not alter the pH of the gastric fluid. In certain embodiments, the alkaline agent is present in an amount from about 30% to about 90% w/w of total Triggering Granules. In certain embodiments, the alkaline agent is present in an amount from about 35% to about 85%, about 40% to about 80%, about 45% to about 75%, about 50% to about 70%, or about 55% to about 65% w/w of total Triggering Granule. In certain embodiments, the alkaline agent is present in an amount from about 40% to about 70%, about 70% to about 90%, or about 50% to about 60%, w/w of the total Triggering Granule.

In certain embodiments, the pH-stabilizing agents for use in the Triggering Granules include, but are not limited to, bismuth aluminate, bismuth carbonate, bismuth subcarbonate, bismuth subgallate, bismuth subnitrate, calcium phosphate, dibasic calcium phosphate, dihydroxyaluminum aminoacetate, dihydroxyaluminum glycine, magnesium glycinate, sodium potassium tartrate, tribasic sodium phosphate, tricalcium phosphate, and combinations thereof. In certain embodiments, the pH-stabilizing agent is a combination of dibasic calcium phosphate/tricalcium phosphate. In certain embodiments, the ratio of dibasic calcium phosphate to tricalcium phosphate (i.e., dibasic calcium phosphate:tricalcium phosphate) is about 1:1 to about 1:5 wt % ratio. In certain embodiments, the ratio of dibasic calcium phosphate to tricalcium phosphate is about 1:1.25 to about 1:4.75, about 1:1.5 to about 1:4.5, about 1:1.75 to about 1:4.25, about 1:2 to about 1:4, about 1:2.25 to about 1:3.75, about 1:2.5 to about 1:3.5, or about 1:2.75 to about 1:3.25 wt % ratio. In certain embodiments, the pH-stabilizing agent is anhydrous dibasic calcium phosphate.

In certain embodiments, the pH-stabilizing agent is present in an amount that when a single dosage unit is taken, it does not alter the pH of the gastric fluid, but when multiple dosage units are taken (e.g., three or more dosage units), the pH-stabilizing agent maintains the elevated pH levels caused by the alkaline agent. In certain embodiments, the pH-stabilizing agent is present in an amount sufficient to maintain or stabilize the pH of the gastric fluid above about 5.0 for up to five hours. In certain embodiments, the pH-stabilizing agent is present in an amount sufficient to maintain the pH of the gastric fluid above about 5.0 for about one to about two hours. In certain embodiments, the pH-stabilizing agent is present in an amount sufficient to maintain the pH of the gastric fluid above about 5.0 for at least about 1 hour, at least about 1.25 hours, at least about 1.5 hours, at least about 1.75 hours, at least about 2 hours, at least about 2.25 hours, at least about 2.5 hours, at least about 2.75 hours, at least about 3 hours, at least about 3.25 hours, at least about 3.5 hours, at least about 3.75 hours, at least about 4 hours, at least about 4.25 hours, at least about 4.5 hours, at least about 4.75 hours, at least about 5 hours.

In certain embodiments, the pH-stabilizing agent is present in an amount from about 10% to about 60% w/w of total Triggering Granules. In certain embodiments, the pH-stabilizing agent is present in an amount from about 12.5% to about 57.5%, about 15% to about 55%, about 17.5% to about 52.5%, about 20% to about 50%, about 22.5% to about 47.5%, about 25% to about 45%, about 27.5% to about 42.5%, about 30% to about 40%, or about 32.5% to about 37.5% w/w of total Triggering Granules. In certain embodiments, the pH-stabilizing agent is present in an amount from about 15% to about 40%, or about 20% or about 30%, w/w of total Triggering Granules.

In certain embodiments, the alkaline agent and the pH-stabilizing agent (combined) (e.g., included in the Triggering Particulates) are present in an amount of less than 60% w/w (i.e., 60 wt %) of the total dosage form (or pharmaceutical composition). In certain embodiments, the alkaline agent and the pH-stabilizing agent (combined) are present in an amount of less than 60%, less than 55%, less than 50%, less than 45%, less than 44%, less than 43%, less than 42%, less than 41%, less than 40%, less than 39%, less than 38%, less than 37%, less than 36%, less than 35%, less than 34%, less than 33%, less than 32%, less than 31%, less than 30%, less than 29%, less than 28%, less than 27%, less than 26%, less than 25%, less than 24%, less than 23%, less than 22%, less than 21%, less than 20%, less than 19%, less than 18%, less than 17%, less than 16%, or less than 15%, w/w of the total dosage form.

In certain embodiments, the Triggering Granules include a binder, a disintegrant, filler (or diluents), and/or a lubricant.

Binders according to the present invention include, but are not limited to, hydroxypropyl celluloses in various grades, hydroxypropyl methylcelluloses in various grades, polyvinylpyrrolidones in various grades, copovidones, powdered acacia, gelatin, guar gum, carbomers, methylcelluloses, polymethacrylates, and starches.

Disintegrants according to the present invention include, but are not limited to, carmellose calcium, carboxymethylstarch sodium, croscarmellose sodium, crospovidone (crosslinked homopolymer of N-vinyl-2-pyrrolidone), low-substituted hydroxypropyl celluloses, sodium starch glycolate, colloidal silicon dioxide, alginic acid and alginates, acrylic acid derivatives, and various starches.

Lubricants according to the present invention include, but are not limited to, magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and any combinations thereof.

The Triggering Granules may be prepared by any granulation method known to those of skill in the art. For example, the Triggering Granules can be made by dry granulation (e.g., direct blend, compacting and densifying the powders), wet granulation (e.g., addition of a granulation liquid onto a powder bed under the influence of an impeller or air), or hot melt extrusion (HME). The granulation product obtained can be milled to achieve uniform granules. The granules obtained may be subsequently coated with an aqueous dispersion.

In certain embodiments, the mean particle size distribution of the Triggering Granules is about 100 μm to about 1000 μm. In certain embodiments, the mean particle size distribution of the Triggering Granules is about 150 μm to about 950 μm, about 200 am to about 900 μm, about 250 μm to about 850 μm, about 300 μm to about 800 μm, about 350 μm to about 750 μm, about 400 μm to about 700 μm, about 450 μm to about 650 μm, or about 500 μm to about 600 μm. In certain embodiments, the mean particle size distribution of Triggering Granules is about 300 μm to about 800 μm.

### 5.4. Viscosity Enhancing Particulates

In certain embodiments, the Viscosity Enhancing Particulates can be Viscosity Enhancing Granules. Viscosity Enhancing Granules increase the viscosity of the dosage form when added to a dissolution medium (e.g., water), thus impeding the ability to extract the active agent from the dosage form, or to pass the dissolution medium with the active agent through a needle for injection purposes.

In certain embodiments, the increase in viscosity may also reduce the potential absorption of the active agent when taken in amounts in excess of two dosage units (e.g., three or more dosage units). As the viscosity of the solution in the GI tract increases, the active agent is eventually entrapped in a polymer gel matrix and the dosage form is transformed from an immediate release formulation to the equivalent of an extended release formulation. It is believed that the ingestion of increasing quantities of the formulation will not proportionally increase the maximum concentration (Cₘₐₓ) to reach the full potential of abusive effects (e.g., euphoria, sedation, and/or relaxation) of the active agent. In addition, it will take a longer time to reach maximum concentration (Tₘₐₓ). The result will be a reduced desirability of deliberately abusing or overdosing on the active agent.

In certain embodiments, the Viscosity Enhancing Granules contain a viscosity-building polymer. In certain embodiments, the viscosity-building polymer is present in an amount that is sufficient to increases the viscosity of the proximal fluid in the GI tract if multiple doses, e.g., three or more dosage units, are taken, e.g., deliberately for the purpose of abuse. In certain embodiments, the viscosity-building polymer is present in an amount that prevents syringeability by rapidly forming a gelatinous mass that resists passage through a needle when one or more units are subjected to incubation in about 10 ml of aqueous or nonaqueous media.

In certain embodiments, the Viscosity Enhancing Granules include a polymer matrix that may include a nonionic polymer (e.g., polyethylene oxide (PEO) polymers such as Polyox® WSR coagulant, Polyox® WSR-301, Polyox® WSR-303) and/or pH-dependent polymers (e.g., carbomers such as Carbopol 934P, Carbopol 971P, Carbopol 974P).

In certain embodiments, Viscosity Enhancing Granules include an antioxidant, a plasticizer, and/or a surfactant, each of which may be the same or different from those used in the Active Granules. In certain embodiments, the Viscosity Enhancing Granules matrix further includes a glidant (e.g., talc, colloidal silicon dioxide, magnesium trisilicate, powdered cellulose, starch, and tribasic calcium phosphate). In certain embodiments, the Viscosity Enhancing Granules matrix further includes a disintegrant, which may be the same or different from those used in the Triggering Granules.

In certain embodiments, the viscosity-building polymer is present in an amount that does not retard the release of the active agent from a single dose administration, but does slow down the release of the active agent when multiple dosage units are taken together (e.g., three or more dosage units). In certain embodiments, the viscosity-building polymer is present in an amount from about 2% to about 60% w/w of total Viscosity Enhancing Granules. In certain embodiments, the viscosity-building polymer is present in an amount from about 5% to about 55%, about 10% to about 50%, about 15% to about 45%, about 20% to about 40%, or about 25% to about 35% w/w of total Viscosity Enhancing Granules. In certain embodiments, the viscosity-building polymer is present in an amount from about 10% to about 50%, or about 15% to about 20%, w/w of total Viscosity Enhancing Granules.

Viscosity Enhancing Granules may be prepared by any granulation method known to those of skill in the art. For example, the Viscosity Enhancing Granules can be made by dry granulation (e.g., direct blend, compacting and densifying the powders), wet granulation (e.g., addition of a granulation liquid onto a powder bed under the influence of an impeller or air), melt granulation, hot-melt extrusion, extrusion spheronization, or rotor granulation. The granulation product obtained can be milled to achieve uniform granules. The granules obtained may be subsequently coated with an aqueous dispersion.

In certain embodiments, the mean particle size distribution of the Viscosity Enhancing Granules is about 125 μm to about 1000 μm. In certain embodiments, the mean particle size distribution of the Viscosity Enhancing Granules is about 150 μm to about 950 μm, about 200 μm to about 900 μm, about 250 μm to about 850 μm, about 300 am to about 800 μm, about 350 μm to about 750 μm, about 400 μm to about 700 μm, about 450 μm to about 650 μm, or about 500 μm to about 600 μm. In certain embodiments, the mean particle size distribution of Viscosity Enhancing Granules is about 250 μm to about 750 μm.

### 5.5. Particulate and Multi-Particulate Dosage Forms

The present invention combines ADF and ODP properties in single solid oral immediate release dosage form and thus addresses multiple health-related concerns, especially regarding habit-forming active agents compounds for which there is a high propensity for abuse (e.g., opioids). In certain embodiments, the abuse deterrence and/or overdose protection activates after the ingestion of three or more dosage units (e.g., three or more tablets/capsules). In certain embodiments, the abuse deterrence and/or overdose protection activates when the multiple dosage units are taken at once. In certain embodiments, the abuse deterrence and overdose protection may activate when the multiple dosage units are taken in tandem. In certain embodiments, release of the active agent after ingesting one to two dosage units results in the dosage form maintaining its (their) immediate release properties (i.e., there is no (or minimal) effect on the release of the active agent from the dosage form(s)). In certain embodiments, if three or more dosage units are taken, release of the active agent from the dosage form is significantly reduced. In certain embodiments, the release is reduced by more than 25%, 30%, 35%, 40%, 45%, 50%, 55%, 60%, 65%, 70%, 75%, 80%, 85%, 90%, 95%, 96%, 97%, 98%, 99%, or increments therein. These dosage forms, however, are not intended to be used as an extended release or sustained release dosage form.

In certain embodiments, the immediate release pharmaceutical dosage form is a particulate dosage form. In certain embodiments, the pharmaceutical dosage forms (multi-particulates) contain at least two different populations of particulates. In certain embodiments, the immediate release pharmaceutical dosage forms contain at least three different populations of particulates. In certain embodiments, the immediate release pharmaceutical dosage forms contain at least four, at least five, at least six, or at least seven different populations of particulates. Each population of particulates is designed for a specific function to accomplish the desired combination of abuse deterrence and overdose protection qualities.

In certain embodiments, the pharmaceutical dosage forms contain at least one population of Active Particulates (e.g., Active Pellets and/or Active Granules) in combination with at least one population of Triggering Granules. In certain embodiments, the alkaline agent of the Triggering Granules increases the pH of the aqueous or nonaqueous solution to above about pH 5.0 in the presence of three or more dosage units, and the pH-stabilizing agent of the Triggering Granules maintains the increased pH above about 5.0 for up to two hours. In certain embodiments, the functional coating of the Active Particulates only allows the release of the active agent in an aqueous or nonaqueous environment with a pH below about 5.0 and prevents or slows the release of the active agent at a pH above about 5.0. In certain embodiments, the pharmaceutical dosage forms contain at least one population of Viscosity Enhancing Granules. In certain embodiments, the pharmaceutical dosage forms contain at least one population of Active Particulates (e.g., Active Pellets and/or Active Granules, comprising, e.g., an opioid(s)) in combination with at least one population of Triggering Granules and at least one population of Viscosity Enhancing Granules. In certain embodiments, the Viscosity Enhancing Granules are present in an amount of from about 2% to about 50% of the total weight of the dosage form.

In certain embodiments, the pharmaceutical dosage forms may contain at least one population of pH-Dependent Viscosity Modifying Particulates. In certain embodiments, pH-dependent Viscosity Modifying Particulates are pH-dependent Viscosity Modifying Granules comprising pH-dependent viscosity building polymer (e.g., carbomers such as Carbopol 934P, Carbopol 971P, and Carbopol 974P). In certain embodiments, the pH-dependent viscosity building polymer may be present in an amount that does not retard the release of the active agent from a single dose administration, but does slow down the release of the active agent after multiple dosage units are taken. In certain embodiments, the pH-dependent Viscosity Modifying Granules may be present in an amount from about 0.5% w/w to about 15% w/w of the total weight of the dosage form. In certain embodiments, the pH-dependent Viscosity Modifying Granules may be present in an amount from about 0.75% w/w to about 12.5%, about 1% to about 10%, or about 2.5% to about 7.5% w/w of the total weight of the dosage form.

In certain embodiments, the pharmaceutical dosage forms contain at least one population of pH-Dependent Viscosity Modifying Granules. In certain embodiments, the pharmaceutical dosage forms contain at least one population of Active Particulates in combination with at least one population of Triggering Granules and at least one population of pH-Dependent Viscosity Modifying Granules. In certain embodiments, the pharmaceutical dosage forms contain at least one population of Active Particulates in combination with at least one population of Triggering Granules, at least one population of Viscosity Enhancing Granules, and at least one population of pH-Dependent Viscosity Modifying Granules.

In certain embodiments, the pharmaceutical dosage forms may contain at least one population of Ion Exchange Resin Granules (e.g., Amberlite™ IRP 64, Amberlite™ IRP 69). The ion exchange resins of the Ion Exchange Resin Granules form a matrix or complex with the drug, and thus may alter the release of drug. In certain embodiments, the ion exchange resin may be present in an amount that binds to the active agent if the dosage form is tampered with, thereby preventing the release of the active agent from the dosage form. In certain embodiments, the Ion Exchange Resin Granules may be present in a concentration of about 1-5 M and in some embodiments from about 1-3 M, based on the total molarity of the drug susceptible to abuse.

In certain embodiments, the pharmaceutical dosage forms contain at least one population of Ion Exchange Resin Granules. In certain embodiments, the pharmaceutical dosage forms contain at least one population of Active Particulates in combination with at least one population of Triggering Granules and at least one population of Ion Exchange Resin Granules. In certain embodiments, the pharmaceutical dosage forms contain at least one population of Active Particulates in combination with at least one population of Triggering Granules, at least one population of Viscosity Enhancing Granules, and at least one population of Ion Exchange Resin Granules. In certain embodiments, the pharmaceutical dosage forms contain at least one population of Active Particulates in combination with at least one population of Triggering Granules, at least one population of Viscosity Enhancing Granules, at least one population of pH-Dependent Viscosity Modifying Granules, and at least one population of Ion Exchange Resin Granules.

In certain embodiments, the pharmaceutical dosage forms contain at least one population of Active Particulates and Triggering Particulates.

In certain embodiments, the AD and ODP characteristics of the dosage form have a synergistic effect(s). In certain embodiments, ODP elements of the dosage form further enhance AD features of the dosage form, i.e., in a synergistic manner. In certain embodiments, AD elements of the dosage form further enhance ODP features of the dosage form, i.e., in a synergistic manner. In certain embodiments, the ODP elements, e.g., acid labile coat (functional coat) on the Active Particulates, and/or the presence of alkaline agent in, e.g., Triggering Particulates, enhance the AD features (e.g., reduce the amount of active in the syringeable liquid by further controlling the release of the active agent from the dosage form in certain embodiments of deliberate abuse).

In certain embodiments, the pharmaceutical dosage form of the invention is a solid immediate release multi-particulate dosage form with abuse deterrent properties and overdose protection elements, comprising a first population of particulates comprising a therapeutically effective amount of at least one opioid embedded in a polymer matrix, and an acid labile coat, and a second population of particulates comprising an alkaline agent, wherein the abuse deterrent properties comprise resistance to extractability, and resistance to syringeability of the opioid; and the ODP elements comprise the acid labile coat, and an alkaline agent; wherein the presence of overdose protection elements enhance the abuse deterrent properties of the dosage form in a synergistic manner. In certain embodiments, the presence of the alkaline agent reduces the amount of active agent present in a syringeable liquid to less than about 10-20%, compared with about 40% of the opioid in a dosage form without an alkaline agent. In certain embodiments, the syringeable liquid is obtained by adding at least one crushed dosage form, with or without an alkaline agent, to water at room temperature and maintaining the resulting suspension at room temperature for, e.g., 30 minutes. In certain embodiments, the dosage form without an alkaline agent comprises a single population of particulates comprising a therapeutically effective amount of at least one opioid embedded in a polymer matrix, and an acid labile coat. In certain embodiments, the dosage form without an alkaline agent comprises a tablet dosage form without Triggering Particulates.

In certain embodiments, the pharmaceutical dosage form of the invention is a solid immediate release multi-particulate dosage form with abuse deterrent properties and an overdose protection element, comprising a population of particulates comprising a therapeutically effective amount of at least one opioid embedded in a polymer matrix, and an acid labile coat; wherein the abuse deterrent properties comprise resistance to extractability, and resistance to syringeability of the opioid; and the ODP element comprises the acid labile coat; wherein the presence of the overdose protection element enhances the abuse deterrent properties of the dosage form in a synergistic manner. In certain embodiments, the presence of the acid labile coat on the particulates reduces the amount of active agent present in the syringeable liquid to less than about 10-20%, compared with about 40% of the opioid in a dosage form comprising particulates without an acid labile coat. In certain embodiments, the acid labile coat comprises a cationic polymer, e.g., a copolymer based on dimethylaminoethyl methacrylate, butyl methacrylate, and methyl methacrylate, that dissolves at a pH of less than about 5.0. In certain embodiments, the syringeable liquid is obtained by adding at least one crushed dosage form, with or without an alkaline agent, to water at room temperature and maintaining the resulting suspension at room temperature for, e.g., five minutes. In certain embodiments, the dosage form without an acid labile coat comprises a population of particulates comprising a therapeutically effective amount of at least one opioid embedded in a polymer matrix. In certain embodiments, the dosage form without an acid labile coat comprises a tablet dosage form without an acid labile coating on the Active Particulates.

In certain embodiments, the alkaline agent present in Triggering Particulates increases the viscosity of the dosage form by activating pH-dependent anionic polymer(s), e.g., gelling polymers such as carbomers, thereby enhancing the AD features (AD properties), such as reduced dissolution and syringeability of the dosage form, in a synergistic manner. In certain embodiments, the gelling effect of, e.g., carbomers is greatly enhanced in the raised pH resulting from the alkaline agent released from the Triggering Granules involved in ODP. The increased AD effects of such gelling can be part of, e.g., decreases in attempted extraction, and decreased release of active agent in the stomach when three or more dosage units are ingested.

In certain embodiments, the plurality of particulate populations can be blended with other excipients and additives and compressed into a tablet or loaded into a capsule. In certain embodiments, the tablet/capsule dosage form disintegrates rapidly once in contact with aqueous medium. In certain embodiments, the capsule may be a soft or hard gelatin capsule. In certain embodiments, the capsule itself does not alter the release of the active agent.

In certain embodiments, Active Particulates are present in an amount from about 10% to about 80% w/w of the total weight of the dosage form. In certain embodiments, the Active Particulates are present in an amount from about 15% to about 75%, about 20% to about 70%, about 25% to about 65%, about 30% to about 60%, about 35% to about 55%, or about 40% to about 50% w/w of the total weight of the dosage form. In certain embodiments, the Active Particulates are present in an amount from about 50% to about 80%, about 60% to about 80%, or about 70% to about 80% w/w of the total weight of the dosage form. In certain embodiments, the Active Particulates are present in an amount from about 10% to about 70%, about 20% to about 70%, about 30% to about 70%, or about 40% to about 70% w/w of the total weight of the dosage form. In certain embodiments, the Active Particulates are present in an amount of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, or at least about 80% w/w of the total weight of the dosage form.

In certain embodiments, the Triggering Granules are present in an amount from about 10% to about 50% w/w of the total weight of the dosage form. In certain embodiments, the Triggering Granules are present in an amount from about 20% to about 42% w/w of the total weight of the dosage form. In certain embodiments, the Triggering Granules are present in an amount from about 22% to about 40%, about 24% to about 38%, about 26% to about 36%, about 28% to about 34%, or about 30% to about 32% w/w of the total weight of the dosage form. In certain embodiments, the Triggering Granules are present in an amount from about 20% to about 42%, about 22% to about 42%, about 24% to about 42%, about 26% to about 42%, about 28% to about 42%, about 30% to about 42%, about 32% to about 42%, about 34% to about 42%, about 36% to about 42%, about 38% to about 42%, or about 40% to about 42% w/w of the total weight of the dosage form. In certain embodiments, the Triggering Granules are present in an amount of at least about 20%, at least about 22%, at least about 24%, at least about 26%, at least about 28%, at least about 30%, at least about 32%, at least about 34%, at least about 36%, at least about 38%, at least about 40%, or at least about 42% w/w of the total weight of the dosage form.

In certain embodiments, the Viscosity Enhancing Granules are present in an amount from about 2% to about 50% w/w of the total weight of the dosage form. In certain embodiments, the Viscosity Enhancing Granules are present in an amount from about 5% to about 45%, about 10% to about 40%, about 15% to about 35%, or about 20% to about 30% w/w of the total weight of the dosage form.

In certain embodiments, the pH-Dependent Viscosity Modifying Granules are present in an amount from about 0.5% to about 15% w/w of the total weight of the dosage form. In certain embodiments, the pH-Dependent Viscosity Modifying Granules are present in an amount from about 0.75% to about 12.5%, about 1% to about 10%, or about 2.5% to about 7.5% w/w of the total weight of the dosage form.

In certain embodiments, the Ion Exchange Resin Granules are present in a concentration of about 1-5 M, or about 1-3 M, based on the total molarity of the drug susceptible to abuse.

In certain embodiments, a single particulate population (e.g., a population of Opioid Particulates) can be blended with other excipients and additives and compressed into various tablet dosage forms, e.g., tablet, mini-tablet, tablet-in-tablet, bilayer tablet, or multilayer tablet, or loaded into a capsule, or the like. In certain embodiments, additional solid IR dosage forms, including additional particulate, tablet, and/or capsule coating regimens, are contemplated. A nonlimiting set of exemplary dosage forms follows.

In certain embodiments, the formulation is a single particulate dosage form comprising a single population of particulates (e.g., comprising a functional coat) containing at least one opioid, the particulates being compressed into a tablet/mini-tablet or filled in a capsule, and at least one alkalinizing coat covering the tablet/mini-tablet and/or capsule.

In certain embodiments, the multi-particulate dosage form is a two particulate dosage form comprising a first population of Active Particulates containing an opioid, and a second population of Triggering Particulates, the two particulate populations being compressed into a tablet/mini-tablet or filled in a capsule.

In certain embodiments, the tablet/mini-tablet is further coated with an acid labile coat and, optionally, an alkalinizing coat on top of the acid labile coat.

In certain embodiments, Active Particulates contain an alkaline agent and, optionally, a pH-stabilizing agent in the polymer matrix.

In certain embodiments, the size of Active Particulates is, e.g., about 400 micrometers to about 2-3 mm, to provide enhanced control of release of active agent (e.g., opioid) in an ODP setting, while providing required and desired immediate release (independent of any food effect) when one or two dosage units are consumed.

In certain embodiments, the Active Particulates can have various functional coat layer(s) (e.g., without limitation, FC 0, FC 1, or FC 2, or combinations thereof).

In certain embodiments, the Active Particulates have a seal coat (optional) on top of the polymer matrix.

In certain embodiments, the Active Particulates have an over coat on top of the functional coat layer(s).

In certain embodiments, capsules contain coated Active Particulates (e.g., Opioid Particulates) coated with a functional coat layer(s) and an over coat, and Triggering Particulates.

In certain embodiments, capsules contain Triggering Particulates, and tablets/mini-tablets made from coated Active Particulates.

In certain embodiments, capsules contain tablets/mini-tablets of coated Active Particulates, and tablets/mini-tablets of Triggering Particulates.

In certain embodiments, capsules contain coated Active Particulates, and tablets/mini-tablets of Triggering Particulates.

In certain embodiments, capsules contain (1) mini-tablets/tablets comprising coated Active Particulates, and at least a portion of Triggering Particulates; and (2) a remaining portion of Triggering Particulates.

In certain embodiments, the dosage form is a bilayer tablet comprising a first layer comprising coated Active Particulates, and a second layer comprising Triggering Particulates, and the two layers are compressed into a bilayer tablet. In certain embodiments, the first layer is coated with at least one functional coat layer and an over coat on top of the at least one functional coat layer.

In certain embodiments, the dosage form is a bilayer tablet comprising a first layer comprising a coated tablet comprising Active Particulates, and a second layer comprising Triggering Particulates, and the two layers are compressed into a bilayer tablet.

In certain embodiments, the dosage form is a tablet-in-tablet dosage form comprising an inner tablet comprising coated Active Particulates, and an outer tablet, comprising Triggering Particulates, encasing the inner tablet.

In certain embodiments, the dosage form is a tablet-in-tablet dosage form comprising an inner coated tablet comprising Active Particulates, and an outer tablet, partially or completely encasing the inner tablet, comprising Triggering Particulates.

In certain embodiments, the dosage form is a capsule dosage form comprising Triggering Particulates, and compressed tablets/mini-tablets comprising Active Particulates (e.g., Opioid Particulates).

In certain embodiments, the dosage form is a capsule dosage form comprising Active Particulates (e.g., Opioid Particulates), and compressed tablets/mini-tablets comprising Triggering Particulates.

In certain embodiments, the dosage form is a capsule dosage form comprising compressed tablets/mini-tablets comprising Active Particulates (e.g., Opioid Particulates), and compressed tablets/mini-tablets comprising Triggering Particulates.

### 5.6. Syringeability and Extractability Resistance, and Heat Stability

In certain embodiments, the particulate and multi-particulate dosage forms of the present invention provide several additional abuse-deterrent properties, including syringeability resistance, extractability resistance, and heat stability. For example, the multi-particulate dosage forms resist abuse via, but not limited to, extraction of the opioid from the dosage form, syringeability of the opioid from the dosage form, and destabilization of the several abuse-deterrent attributes by various thermal pretreatment-related manipulations (e.g., heating or freezing of the dosage form before mechanical manipulations, e.g., crushing or grinding). In certain embodiments, the combination of these additional properties, along with the aforementioned resistance to crushability and grindability of the Opioid Particulates, strongly deter or prevent abuse of the inventive multi-particulate dosage form.

In certain embodiments, resistance to extractability is provided by, e.g., carbomers in the Opioid Particulates of the dosage form. In certain embodiments, carbomers (such as Carbopol 934P, Carbopol 971P, Carbopol 974P), as well as other anionic polymers that are viscosity-enhancing agents, form gel and increase viscosity in aqueous and/or alcoholic media, such as those media used by abusers attempting extraction of opioid from a given dosage form. In certain embodiments, the gelling effect of carbomers is greatly enhanced in alkaline pH resulting from the alkaline agent released from the Triggering Granules (e.g., in attempted extraction, or in the stomach when three or more dosage units are ingested), or the alkaline agent when present in the polymer matrix. In certain embodiments, carbomers in the core form gel and further diminish drug release, e.g., permeation from the core of Opioid Particulates into the GI fluid, or into aqueous media attempting to be drawn into a syringe. In certain embodiments, polymers present in the functional coat(s), e.g., EUDRAGIT® E PO, are also involved in decreasing permeation of the opioid from the Opioid Particulates, e.g., when extraction is attempted. The alkaline agent(s) present in the dosage forms produce a rapid rise in the pH of aqueous media (e.g., in attempted extraction, or in the stomach when three or more dosage units are ingested). The polymers present in the functional coats, e.g., EUDRAGIT® E PO, become insoluble in this alkaline media; thus the release of opioid from the dosage form is blocked.

In certain embodiments, resistance to syringeability is provided by polyoxyethylene (PEO) polymers and HPMC in the Opioid Particulates (e.g., in the core of the Opioid Granules). The gelling characteristics of these molecules, when exposed to aqueous media, provide resistance to syringeability as the bore of the needle is blocked by the viscous nature of the diluted dosage form. In addition, carbomers included in the dosage form (e.g., in the core of the Opioid Granules) provide further resistance to syringeability; in response to the rapidly rising pH induced by, e.g., Mg(OH)₂ in aqueous media, carbomer-based gelling is greatly enhanced, further diminishing drug release. In certain embodiments, carbomers included in the dosage form (e.g., in the core of the Opioid Granules) provide further resistance to syringeability in response to the rising pH induced by the interaction of aqueous media with Mg(OH)₂ present in the core. Thus, less drug permeates into the aqueous media, and less drug is available to be drawn into the syringe. In certain embodiments, polymers present in the functional coats, e.g., EUDRAGIT® E PO, are also involved in resistance to syringeability. The alkaline agent(s) present in the dosage form produces a rapid rise in the pH of aqueous media. The polymers present in the functional coats, e.g., EUDRAGIT® E PO, become insoluble in this alkaline media and block release of opioid from the dosage form. Thus, attempts to draw fluid containing the opioid into a syringe are blocked in this manner as well.

In certain embodiments, resistance to syringeability and extractability are provided by one or more properties of the dosage form. For example, resistance is provided by the gelling characteristics of polyoxyethylene (PEO) polymers and HPMC in the Opioid Particulates (e.g., in the core of the Opioid Granules) when exposed to aqueous media; such gelling results in less drug permeating into the aqueous media, and less drug being available to be drawn into a syringe. In addition, carbomers and alkaline agent(s) included in the matrix core of the dosage form (e.g., in the core of the Opioid Particulates) provide further resistance to syringeability; in response to the rapidly rising pH induced by Mg(OH)₂ in aqueous media; carbomer-based gelling is greatly enhanced, diminishing drug release. Also in response to the elevated pH induced by Mg(OH)₂ (present in the Triggering Particulates), the functional coats remain relatively intact, further diminishing drug release from the dosage form. These unique combinations of elements and features of the dosage form are prominent, for example, in a physiological setting involving accidental overdose (or deliberate abuse) comprising ingestion of multiple dosage units (dosage forms).

The following examples are offered to more fully illustrate the invention, but are not to be construed as limiting the scope thereof.

### 6. EXAMPLES

### Example 1: Crush-Resistant Oxycodone Hydrochloride Granule Cores (Active Granules)

Oxycodone hydrochloride granule cores were prepared for use in a 5 mg, 10 mg, 15, mg, and 30 mg oxycodone hydrochloride dosage form.

TABLE 1
Formulation of Active Granule Cores

| | Active | Active | Active | Active |
|---|---|---|---|---|
| | Granule | Granule | Granule | Granule |
| | Core 1 | Core 2 | Core 3 | Core 4 |
| Components | mg/dose | mg/dose | mg/dose | mg/dose |
| Oxycodone | 5.00 | 10.00 | 15.00 | 30.00 |
| hydrochloride | | | | |
| Polyethylene oxide | 65.44 | 65.44 | 65.44 | 50.44 |
| (POLYOX⁽™⁾) | | | | |
| Microcrystalline | 10.00 | 5.00 | NA | NA |
| Cellulose (Avicel PH | | | | |
| 101) | | | | |
| Hypromellose (Benecel | 9.41 | 9.41 | 9.41 | 9.41 |
| K200M) | | | | |
| Kollidon SR | 4.71 | 4.71 | 4.71 | 4.71 |
| Triethyl citrate | 3.24 | 3.24 | 3.24 | 3.24 |
| Docusate sodium (85%) | 2.00 | 2.00 | 2.00 | 2.00 |
| with sodium benzoate | | | | |
| (15%) (DOSS) | | | | |
| Vitamin E (dl-α- | 0.20 | 0.20 | 0.20 | 0.20 |
| Tocopherol) | | | | |
| Total | 100 | 100 | 100 | 100 |

### Manufacturing Procedure:

1. Oxycodone hydrochloride, polyethylene oxide, microcrystalline cellulose, hypromellose, Kollidon SR, and docusate sodium were added to a high shear granulator and mixed into a uniform powder mix using an impeller and a chopper.
2. A solution of dl-α-tocopherol solution and triethyl citrate was sprayed onto the powder mix from step #1 to achieve a uniform blend.
3. The blend from step #2 was granulated by hot-melt extrusion.
4. The granules from step #3 were processed using cryomilling to a mean particle size of about 500 μm.

### Example 2: Crush-Resistant Hydromorphone Hydrochloride Granule Cores (Active Granules)

Hydromorphone hydrochloride granule core was prepared for use in an 8 mg hydromorphone hydrochloride dosage form.

TABLE 2
Formulation of Active Granule Cores

| Components | mg/dose |
|---|---|
| Hydromorphone hydrochloride | 8.00 |
| Polyethylene oxide (POLYOX⁽™⁾) | 32.20 |
| Hypromellose (Benecel K 200M) | 4.71 |
| Kollidon ® SR | 2.36 |
| Triethyl citrate | 0.10 |
| Docusate sodium | 1.62 |
| Vitamin E (dl-α-Tocopherol) | 1.00 |
| Total | 50.00 |

### Manufacturing Procedure:

1. Hydromorphone hydrochloride, polyethylene glycol, hypromellose, Kollidon® SR, and docusate sodium were added to a high shear granulator and mixed into a uniform powder mix using an impeller and a chopper.
2. A solution of dl-α-tocopherol solution and triethyl citrate was sprayed onto the powder mix from step #1 to achieve a uniform blend.
3. The blend from step #2 was granulated by hot-melt extrusion.
4. The granules from step #3 were processed using cryomilling to a mean particle size of about 500 μm.

### Example 3: Crush-Resistant Hydrocodone Bitartrate Granule Cores (Active Granules)

Hydrocodone bitartrate granule core was prepared for use in a 10 mg hydrocodone bitartrate dosage form.

TABLE 3
Formulation of Active Granule Cores

| Components | mg/dose |
|---|---|
| Hydrocodone bitartrate | 10.00 |
| Polyethylene oxide (POLYOX⁽™⁾) | 70.44 |
| Hypromellose (Benecel K 200M) | 9.41 |
| Kollidon ® SR | 4.71 |
| Triethyl citrate | 0.20 |
| Docusate sodium | 3.24 |
| dl-α-Tocopherol | 2.00 |
| Total | 100.00 |

### Manufacturing Procedure:

1. Hydrocodone bitartrate, polyethylene oxide, hypromellose, Kollidon® SR, and docusate sodium are added to a high shear granulator and mixed into a uniform powder mix using an impeller and a chopper.
2. A solution of dl-α-tocopherol solution and triethyl citrate is sprayed onto the powder mix from step #1 to achieve a uniform blend.
3. The blend from step #2 is granulated by hot-melt extrusion.
4. The granules from step #3 are processed cryomilling to a mean particle size of about 500 μm.

### Example 4: Crush-Resistant Oxymorphone Hydrochloride Granule Cores (Active Granules)

Oxymorphone hydrochloride granule cores are prepared according to procedures similar to those in Examples 1-3.

### Example 5: Seal Coating of Oxycodone Hydrochloride Granule Cores

Oxycodone hydrochloride active granule cores were coated with a seal coat.

TABLE 4
Formulation of Seal Coated Granules

| | Seal | Seal | Seal | Seal |
|---|---|---|---|---|
| | Coated | Coated | Coated | Coated |
| | Granule 1 | Granule 2 | Granule 3 | Granule 4 |
| Components | mg/dose | mg/dose | mg/dose | mg/dose |
| Active Granule Cores | 100.00 | 100.00 | 100.00 | 100.00 |
| (Oxycodone | | | | |
| hydrochloride) | | | | |
| Hypromellose (Methocel | 17.78 | 17.78 | 17.78 | 17.78 |
| E5 Premium LV) | | | | |
| Triethyl citrate | 1.78 | 1.78 | 1.78 | 1.78 |
| Colloidal silicon dioxide | 0.44 | 0.44 | 0.44 | 0.44 |
| (Cab-O-Sil (M-5P) | | | | |

Solvent system for coating

| | | | | |
|---|---|---|---|---|
| Purified water | NA | NA | NA | NA |
| Dehydrated alcohol | NA | NA | NA | NA |
| Total | 120.00 | 120.00 | 120.00 | 120.00 |

### Coating Procedure:

1. Hypromellose was added to dehydrated alcohol in a stainless steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°-50° C., and sufficient air volume for fluidization.
5. When the product temperature reached 30° C., the dispersion from step #3 was sprayed onto the granules while maintaining the product temperature of 28°-30° C. and sufficient air volume for the fluidization, until the target coating weight gain (20 mg) was achieved.
6. The coated granules from step #5 were dried.

### Example 6: Seal Coating of Hydromorphone Hydrochloride Granule Cores

Hydromorphone hydrochloride active granule cores were coated with a seal coat.

TABLE 5
Formulation of Seal Coated Granules

| | Seal Coated Granules |
|---|---|
| Components | (mg/dose) |
| Active Granule cores | 50.00 |
| (Hydromorphone | |
| hydrochloride) | |
| Hypromellose (Methocel | 8.89 |
| E5 Premium LV) | |
| Triethyl citrate | 0.89 |
| Colloidal silicon dioxide | 0.22 |
| (Cab-O-Sil (M-5P) | |

Solvent system for coating

| | |
|---|---|
| Purified water | NA |
| Dehydrated alcohol | NA |
| Total | 60.00 |

### Coating Procedure:

1. Hypromellose was added to dehydrated alcohol in a stainless steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°-50° C., and sufficient air volume for fluidization.
5. When the product temperature reached 30° C., the dispersion from step #3 was sprayed onto the granules while maintaining the product temperature of 28°-30° C. and sufficient air volume for the fluidization, until the target coating weight gain (10 mg) was achieved.
6. The coated granules from step #5 were dried.

### Example 7: Seal Coating of Hydrocodone Bitartrate Granule Cores

Hydrocodone bitartrate active granule cores were coated with a seal coat.

TABLE 6
Formulation of Seal Coated Granules

| | Seal Coated Granules |
|---|---|
| Components | (mg/dose) |
| Active Granule Cores | 100.00 |
| (Hydrocodone bitartrate) | |
| Hypromellose (Methocel | 17.78 |
| E5 Premium LV) | |
| Triethyl citrate | 1.78 |
| Colloidal silicon dioxide | 0.44 |
| (Cab-O-Sil (M-5P) | |

Solvent system for coating

| | |
|---|---|
| Purified water | NA |
| Dehydrated alcohol | NA |
| Total | 120.00 |

### Coating Procedure:

1. Hypromellose was added to dehydrated alcohol in a stainless steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°-50° C., and sufficient air volume for fluidization.
5. When the product temperature reached 30° C., the dispersion from step #3 was sprayed onto the granules while maintaining the product temperature of 28°-30° C. and sufficient air volume for the fluidization, until the target coating weight gain (20 mg) was achieved.
6. The coated granules from step #5 were dried.

### Example 8: Seal Coating of Oxymorphone Hydrochloride Granule Cores

Seal coated oxymorphone hydrochloride active granules are prepared according to procedures similar to those in Examples 5-7.

### Example 9: Functional Coating of Seal Coated Oxycodone Hydrochloride Granules

Seal coated oxycodone hydrochloride granules were coated with a first functional coat layer FC 1 comprising a mixture of rate controlling polymers, e.g., cellulose acetate (CA) and EUDRAGIT® E PO, in a ratio of CA: EUDRAGIT® E PO of 60:40, and a second functional coat layer FC 2 comprising EUDRAGIT® E PO as the sole rate controlling polymer.

TABLE 7
Formulation of Functional Coated Active Granules

| | | | | |
|---|---|---|---|---|
| | Functional | Functional | Functional | Functional |
| | Coated | Coated | Coated | Coated |
| | Granule 1 | Granule 2 | Granule 3 | Granule 4 |
| Components | (mg/dose) | (mg/dose) | (mg/dose) | (mg/dose) |

FC 1

| | | | | |
|---|---|---|---|---|
| Seal coated granules | 120.00 | 120.00 | 120.00 | 120.00 |
| Cellulose acetate (CA | 18.00 | 18.00 | 18.00 | 18.00 |
| 398-10NF/EP) | | | | |
| Amino methacrylate | 12.00 | 12.00 | 12.00 | 12.00 |
| copolymer, NT | | | | |
| (EUDRAGIT ® E PO) | | | | |
| Dibutyl Sebacate | 4.50 | 4.50 | 4.50 | 4.50 |
| Colloidal Silicon | 1.50 | 1.50 | 1.50 | 1.50 |
| Dioxide (Cab-O-Sil | | | | |
| M5P) | | | | |

Solvent system for coating

| | | | | |
|---|---|---|---|---|
| Acetone | NA | NA | NA | |
| Purified water | NA | NA | NA | NA |
| Total | 156.00 | 156.00 | 156.00 | 156.00 |

FC 2

| | | | | |
|---|---|---|---|---|
| FC 1 coated granules | 156.00 | 156.00 | 156.00 | 156.00 |
| Amino methacrylate | 72.00 | 72.00 | 72.00 | 72.00 |
| copolymer, NF | | | | |
| (EUDRAGIT ® E PO) | | | | |
| Polyethylene Glycol, | 7.20 | 7.20 | 7.20 | 7.20 |
| NF (Polyglykol 6000 | | | | |
| PF) | | | | |
| Talc USP (2755) | 14.40 | 14.40 | 14.40 | 14.40 |

Solvent system for coating

| | | | | |
|---|---|---|---|---|
| Acetone | NA | NA | NA | NA |
| Purified water | NA | NA | NA | NA |
| Total | 249.6 | 249.6 | 249.6 | 249.6 |

### Coating Procedure:

1. EUDRAGIT® E PO was added to acetone in a stainless steel container and mixed until a clear solution formed.
2. To the solution from step #1, cellulose acetate was added and mixed until a clear solution formed.
3. The purified water was added to the solution from step #2 and mixed for ˜5 minutes.
4. To the solution from step #3, dibutyl sebacate was added followed by colloidal silicon dioxide and continued mixing until a homogenous dispersion was obtained.
5. The seal coated granules were further coated using a Wurster fluid bed coater with an inlet air temperature of 40°-50° C. and sufficient air volume for fluidization.
6. When the product temperature reached 30° C., the dispersion from step #4 was sprayed onto the seal coated granules while maintaining the product temperature of 28°-30° C. and sufficient air volume for the fluidization, until the target coating weight gain (36 mg) was achieved.
7. The coated granules from step #6 were dried to FC 1 coated granules.

The FC 1 coated granules were further coated with a second functional coat layer (FC 2) as follows:
1. EUDRAGIT® E PO was added to acetone in a stainless steel container and mixed until a clear solution form.
2. The purified water was added to the solution from step #1 and mixed for ˜5 minutes.
3. To the solution from step #3, polyethylene glycol was added followed by talc and mixed until a homogenous dispersion was obtained.
4. The FC 1 coated granules were further coated using a Wurster fluid bed coater with an inlet air temperature of 40°-50° C., and sufficient air volume for fluidization.
5. When the product temperature reached 30° C., the dispersion from step #4 was sprayed onto the FC 1 coated granules while maintaining the product temperature of 28°-30° C. and sufficient air volume for the fluidization, until the target coating weight gain (93.6 mg) was achieved.
6. The coated granules from step #6 were dried to FC 2 coated granules.

### Example 10: Functional Coating of Seal Coated Hydromorphone Hydrochloride Granules

Seal coated hydromorphone hydrochloride granules were coated with a first functional coat layer FC 1 comprising a mixture of rate controlling polymers, e.g., cellulose acetate (CA) and EUDRAGIT® E PO, in a ratio of CA:EUDRAGIT® E PO of 60:40, and a second functional coat layer FC 2 comprising EUDRAGIT E PO as the sole rate controlling polymer.

TABLE 8
Formulation of Functional Coated Active Granules

| | |
|---|---|
| | Functional Coated Granules |
| Components | mg/dose |

FC 1

| | |
|---|---|
| Seal coated hydromorphone hydrochloride | 60.00 |
| granules | |
| Cellulose acetate | 9.00 |
| EUDRAGIT ® E PO | 6.00 |
| Dibutyl sebacate | 2.25 |
| Colloidal silicon dioxide | 0.75 |

Solvent system for coating

| | |
|---|---|
| Acetone | NA |
| Purified water | NA |
| Total | 78.00 |

FC 2

| | |
|---|---|
| FC 1 coated granules | 78.00 |
| EUDRAGIT ® E PO | 36.00 |
| Polyethylene glycol | 3.60 |
| Talc | 7.20 |

Solvent system for coating

| | |
|---|---|
| Acetone | NA |
| Isopropyl alcohol | NA |
| Total | 124.80 |

### Coating Procedure:

1. EUDRAGIT® E PO was added to acetone in a stainless steel container and mixed until a clear solution formed.
2. To the solution from step #1, cellulose acetate was added and mixed until a clear solution formed.
3. The purified water was added to the solution from step #2 and mixed for ˜5 minutes.
4. To the solution from step #3, dibutyl sebacate was added followed by colloidal silicon dioxide and continued mixing until a homogenous dispersion was obtained.
5. The seal coated granules were further coated using a Wurster fluid bed coater with an inlet air temperature of 40°-50° C. and sufficient air volume for fluidization.
6. When the product temperature reached 30° C., the dispersion from step #4 was sprayed onto the seal coated granules while maintaining the product temperature of 28°-30° C. and sufficient air volume for the fluidization, until the target coating weight gain (18 mg) was achieved.
7. The coated granules from step #6 were dried to FC 1 coated granules.

The FC 1 coated granules were further coated with a second functional coat layer (FC 2) as follows:
1. EUDRAGIT® E PO was added to acetone in a stainless steel container and mixed until a clear solution form.
2. Isopropyl alcohol was added to the solution from step #1 and mixed for ˜5 minutes.
3. To the solution from step #3, polyethylene glycol was added followed by talc and mixed until a homogenous dispersion was obtained.
4. The FC 1 coated granules were further coated using a Wurster fluid bed coater with an inlet air temperature of 40°-50° C., and sufficient air volume for fluidization.
5. When the product temperature reached 30° C., the dispersion from step #4 was sprayed onto the FC 1 coated granules while maintaining the product temperature of 28°-30° C. and sufficient air volume for the fluidization, until the target coating weight gain (46.80 mg) was achieved.
6. The coated granules from step #6 were dried to FC 2 coated granules.

### Example 11: Functional Coating of Seal Coated Hydrocodone Bitartrate Granules

Seal coated hydrocodone bitartrate granules were coated with a first functional coat layer FC 1 comprising a mixture of rate controlling polymers, e.g., cellulose acetate (CA) and EUDRAGIT® E PO, in a ratio of CA:EUDRAGIT® E PO of 60:40, and a second functional coat layer FC 2 comprising EUDRAGIT® E PO as the sole rate controlling polymer.

TABLE 9
Formulation of Functional Coated Active Granules

| | |
|---|---|
| | Functional Coated Granules |
| Components | (mg/dose) |

FC 1

| | |
|---|---|
| Seal coated hydrocodone bitartrate granules | 120.00 |
| Cellulose acetate | 18.00 |
| EUDRAGIT ® E PO | 12.00 |
| Dibutyl sebacate | 4.50 |
| Colloidal silicon dioxide | 1.50 |

Solvent system for coating

| | |
|---|---|
| Acetone | NA |
| Purified water | NA |
| Total | 156.00 |

FC 2

| | |
|---|---|
| FC 1 coated granules | 156.00 |
| EUDRAGIT ® E PO | 72.00 |
| Polyethylene glycol | 7.20 |
| Talc | 14.40 |

Solvent System for Coating

| | |
|---|---|
| Acetone | NA |
| Isopropyl alcohol | NA |
| Total | 249.60 |

### Coating Procedure:

1. EUDRAGIT® E PO was added to acetone in a stainless steel container and mixed until a clear solution formed.
2. To the solution from step #1, cellulose acetate was added and mixed until a clear solution formed.
3. Isopropyl alcohol was added to the solution from step #2 and mixed for ˜5 minutes.
4. To the solution from step #3, dibutyl sebacate was added followed by colloidal silicon dioxide and continued mixing until a homogenous dispersion was obtained.
5. The seal coated granules were further coated using a Wurster fluid bed coater with an inlet air temperature of 40°-50° C. and sufficient air volume for fluidization.
6. When the product temperature reached 30° C., the dispersion from step #4 was sprayed onto the seal coated granules while maintaining the product temperature of 28°-30° C. and sufficient air volume for the fluidization, until the target coating weight gain (36 mg) was achieved.
7. The coated granules from step #6 were dried to FC 1 coated granules.

The FC 1 coated granules were further coated with a second functional coat layer (FC 2) as follows:
1. EUDRAGIT® E PO was added to acetone in a stainless steel container and mixed until a clear solution form.
2. The purified water was added to the solution from step #1 and mixed for ˜5 minutes.
3. To the solution from step #3, polyethylene glycol was added followed by talc and mixed until a homogenous dispersion was obtained.
4. The FC 1 coated granules were further coated using a Wurster fluid bed coater with an inlet air temperature of 40°-50° C., and sufficient air volume for fluidization.
5. When the product temperature reached 30° C., the dispersion from step #4 was sprayed onto the FC1 coated granules while maintaining the product temperature of 28°-30° C. and sufficient air volume for the fluidization, until the target coating weight gain (93.60 mg) was achieved.
6. The coated granules from step #6 were dried to FC 2 coated granules.

### Example 12: Functional Coating of Seal Coated Oxymorphone Hydrochloride Granules

Seal coated oxymorphone hydrochloride granules are coated with a first functional coat layer FC 1 comprising a mixture of rate controlling polymers, e.g., cellulose acetate (CA) and EUDRAGIT® E PO, in a ratio of CA:EUDRAGIT® E PO of 60:40, and a second functional coat layer FC 2 comprising EUDRAGIT® E PO as the sole rate controlling polymer, according to procedures similar to those in Examples 9-11.

### Example 13: Over Coating of Functional Coated Oxycodone Hydrochloride Granules

Functional coated oxycodone hydrochloride granules were coated with an over coat.

TABLE 10
Formulation of Over Coated Active Granules

| | Over | Over | Over | Over |
|---|---|---|---|---|
| | Coated | Coated | Coated | Coated |
| | Granule 1 | Granule 2 | Granule 3 | Granule 4 |
| Components | (mg/dose) | mg/dose | mg/dose | mg/dose |
| FC 2 coated granules | 249.6 | 249.6 | 249.6 | 249.6 |
| Hypromellose, USP | 28.00 | 28.00 | 28.00 | 28.00 |
| (Methocel E5 Premium | | | | |
| LV) | | | | |
| Triethyl Citrate, NF | 2.88 | 2.88 | 2.88 | 2.88 |
| Talc, USP (2755) | 5.76 | 5.76 | 5.76 | 5.76 |

Solvent System for Coating

| | | | | |
|---|---|---|---|---|
| Dehydrated alcohol | NA | NA | NA | NA |
| Purified water | NA | NA | NA | NA |
| Total | 286.24 | 286.24 | 286.24 | 286.24 |

### Coating Procedure:

1. Hypromellose was added to dehydrated alcohol in a stainless steel container and mixed to form a uniform dispersion.
2. To the dispersion from step #1, the purified water was added and mixed until a clear solution formed.
3. To the solution from step #2, triethyl citrate was added followed by the addition of talc and mixed to form a homogenous dispersion.
4. The granules were coated using a Wurster fluid bed coater with an inlet air temperature of 40°-50° C., and sufficient air volume for fluidization.
5. When the product temperature reached 30° C., the dispersion from step #3 was sprayed onto the granules while maintaining the product temperature of 28°-30° C. and sufficient air volume for the fluidization, until the target coating weight gain (36.44 mg) was achieved.
6. The coated granules from step #5 were dried.

### Example 14: Over Coating of Functional Coated Hydromorphone Hydrochloride Granules

Functional coated hydromorphone hydrochloride granules were coated with an over coat.

TABLE 11
Formulation of Over Coated Active Granules

| | Over Coated Granules |
|---|---|
| Components | mg/dose |
| Functional coated Hydromorphone | 124.80 |
| Hydrochloride granules | |
| Methocel E5 Premium LV | 14.40 |
| Triethyl citrate | 1.44 |
| Colloidal silicon dioxide | 2.88 |

Solvent System for Coating

| | |
|---|---|
| Purified water | NA |
| Dehydrated alcohol | NA |
| Total | 143.52 |

### Coating Procedure:

1. Methocel was added to dehydrated alcohol in a stainless steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°-50° C., and sufficient air volume for fluidization.
5. When the product temperature reached 30° C., the dispersion from step #3 was sprayed onto the granules while maintaining the product temperature of 28°-30° C. and sufficient air volume for the fluidization, until the target coating weight gain (18.72 mg) was achieved.
6. The coated granules from step #5 were dried.

### Example 15: Over Coating of Functional Coated Hydrocodone Bitartrate Granules

Functional coated Hydrocodone bitartrate granules were coated with an over coat.

TABLE 12
Formulation of Over Coated Active Granules

| | Over Coated Granules |
|---|---|
| Components | (mg/dose) |
| Functional coated hydrocodone | 249.60 |
| bitartrate granules | |
| Methocel E5 Premium LV | 28.80 |
| Triethyl citrate | 2.88 |
| Colloidal silicon dioxide | 5.76 |

Solvent System for Coating

| | |
|---|---|
| Purified water | NA |
| Dehydrated alcohol | NA |
| Total | 287.04 |

### Coating Procedure:

1. Methocel was added to dehydrated alcohol in a stainless steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°-50° C., and sufficient air volume for fluidization.
5. When the product temperature reached 30° C., the dispersion from step #3 was sprayed onto the granules while maintaining the product temperature of 28°-30° C. and sufficient air volume for the fluidization, until the target coating weight gain (37.44 mg) was achieved.
6. The coated granules from step #5 were dried.

### Example 16: Over Coating of Functional Coated Oxymorphone Hydrochloride Granules

Functional coated Oxymorphone hydrochloride granules are coated with an over coat according to procedures similar to those in as described in Examples 13-15.

### Example 17: Active Pellets

Active Pellets with microcrystalline cellulose (MCC) core (cellets) were prepared for use in a 30 mg oxycodone hydrochloride dosage form.

TABLE 13
Formulation of Active Pellets

| | Active Pellets |
|---|---|
| Components | (mg/dose) |
| Microcrystalline cellulose pellets (Cellets) | 300.00 |
| Oxycodone Hydrochloride | 30.00 |
| Methocel E5 premium LV | 20.00 |
| Talc | 3.00 |

Solvent system for coating

| | |
|---|---|
| Purified water | NA |
| Dehydrated alcohol | NA |
| Total | 353.00 |

### Manufacturing Procedure:

1. Oxycodone hydrochloride was added to the dehydrated alcohol in a stainless steel container and mixed until it dispersed uniformly.
2. After the oxycodone was uniformly dispersed, METHOCEL™ was gradually added with continuous mixing to form a uniform dispersion.
3. The purified water was added to the dispersion from step #2 and mixed until a clear solution was obtained.
4. To the solution from step #3, talc was added and mixed for at least 30 minutes or more, until it was dispersed.
5. The microcrystalline cellulose pellets were coated using a Wurster fluid bed coater with an inlet air temperature of 40°-50° C. and sufficient air volume for fluidization.
6. When the product temperature reached 30° C., the dispersion from step #4 was sprayed onto the pellets while maintaining the temperature of 28°-30° C. and sufficient air volume for the fluidization, until the target coating weight gain (53 mg) was achieved.
7. The coated pellets from step #6 were dried.

### Example 18: Seal Coating of Pellets

Active Pellets with MCC core were coated with a seal coat.

TABLE 14
Formulation of Seal Coated Pellets

| | Seal coated Active |
|---|---|
| | Pellets 1 |
| Components | (mg/dose) |
| Active Pellets | 353.00 |
| Methocel E5 premium LV | 15.70 |
| Dibutyl sebacate | 0.80 |
| Talc | 5.50 |

Solvent system for coating

| | |
|---|---|
| Purified water | NA |
| Dehydrated alcohol | NA |
| Total | 375.00 |

### Coating Procedure:

1. Methocel was added to dehydrated alcohol in a stainless steel container and mixed into a uniform dispersion.
2. To the dispersion from step #1, the purified water was added and mixed until a clear solution formed.
3. To the solution from step #2, dibutyl sebacate was added followed by the addition of talc and continued mixing until a homogenous dispersion formed.
4. The pellets were coated using a Wurster fluid bed coater with an inlet air temperature of 40°-50° C., and sufficient air volume for fluidization.
5. When the product temperature reached 30° C., the dispersion from step #3 was sprayed onto the pellets while maintaining the product temperature of 28°-30° C. and sufficient air volume for fluidization, until the target coating weight gain (22 mg) was achieved.
6. The coated pellets from step #5 were dried.

### Example 19: Functional Coating of Pellets (60:40)

Seal coated Active Pellets were coated with a functional coat at a ratio of OPADRY® CA to EUDRAGIT® E PO of 60:40.

TABLE 15
Formulation of Functional Coated Pellets

| | Functional Coated |
|---|---|
| | Active Pellets 1 |
| Components | (mg/dose) |
| Seal coated pellets 1 | 375.00 |
| OPADRY ® cellulose acetate clear | 15.54 |
| EUDRAGIT ® E PO | 10.36 |
| Talc | 9.10 |
| Dibutyl sebacate | 2.60 |

Solvent system for coating

| | |
|---|---|
| Acetone | NA |
| Purified water | NA |
| Total | 412.60 |

### Coating Procedure:

1. EUDRAGIT® E PO was added to acetone in a stainless steel container and mixed until a clear solution formed.
2. To the solution from step #1, OPADRY® cellulose acetate was added and mixed until a clear solution formed.
3. To the solution from step #2, the purified water was added and mixed for ˜5 minutes.
4. To the solution from step #3, dibutyl sebacate was added followed by talc and continued mixing until a homogenous dispersion formed.
5. The seal coated pellets were further coated using a Wurster fluid bed coater with an inlet air temperature of 40°-50° C. and sufficient air volume for fluidization.
6. When the product temperature reached 30° C., the dispersion from step #4 was sprayed onto the seal coated granules and pellets while maintaining the product temperature of 28°-30° C. and sufficient air volume for the fluidization, until the target coating weight gain (37.6 mg) was achieved.
7. The coated pellets from step #6 were dried.

### Example 20: Functional Coating of Pellets (80:20)

Seal coated Active Granules and Pellets are coated with a functional coating at a ratio of OPADRY® cellulose acetate or Kollidon SR to EUDRAGIT® E PO of 80:20.

TABLE 16
Formulation of Functional Coated Pellets

| | Functional Coated | Functional Coated |
|---|---|---|
| | Active Pellets 2 | Active Pellets 3 |
| Components | (mg/dose) | (mg/dose) |
| Seal coated pellets 1 | 375.00 | 375.00 |
| Kollidon | 20.70 | NA |
| OPADRY ® cellulose acetate | NA | 20.70 |
| clear | | |
| EUDRAGIT ® E PO | 5.20 | 5.20 |
| Talc | 9.10 | 9.10 |
| Dibutyl sebacate | 2.60 | 2.60 |

Solvent system for coating

| | | |
|---|---|---|
| Acetone | NA | NA |
| Purified water | NA | NA |
| Total | 412.60 | 412.60 |

### Coating Procedure:

1. EUDRAGIT® E PO was added to acetone in a stainless steel container and mixed until a clear solution formed.
2. To the solution from step #1 OPADRY® Cellulose Acetate/Kollidon was added and mixed until a clear solution formed.
3. The purified water was added to the solution from step #2 and mixed for ˜5 minutes.
4. To the solution from step #3 dibutyl sebacate was added followed by talc and continued mixing until a homogenous dispersion formed.
5. The seal coated granules and pellets are further coated using a Wurster fluid bed coater with an inlet air temperature of 40°-50° C. and sufficient air volume for fluidization.
6. When the product temperature reached 30° C., the dispersion from step #4 was sprayed onto the granules and pellets while maintaining the product temperature of 28°-30° C. and sufficient air volume for the fluidization until the target coating weight gain (37.60 mg) was achieved.
7. The coated pellets from step #6 were dried.

### Example 21: Triggering Granules

Triggering Granules were prepared as described below.

TABLE 17
Formulation of Triggering Granules

| | Triggering Granule 1 | Triggering Granule 2 |
|---|---|---|
| Component | (mg/dose) | (mg/dose) |
| Magnesium | 135.00 | 100.00 |
| hydroxide | | |
| Mannitol | 22.50 | 16.66 |
| Crospovidone | 6.71 | 4.99 |
| Total | 164.21 | 121.65 |

### Manufacturing Procedure:

1. Magnesium hydroxide was added to mannitol, and crospovidone in a high shear granulator and mixed using an impeller and chopper to achieve a uniform blend.
2. The blend from step #1 was granulated by wet granulation using purified water.
3. The granules from step #2 were dried at 40° C. using a forced air oven until the LOD was less than 1%.

### Example 22: Viscosity Enhancing Granules

Viscosity Enhancing Granules were prepared as described below:

TABLE 18
Formulation of Viscosity Enhancing Granules

| | Viscosity | Viscosity | Viscosity |
|---|---|---|---|
| | Enhancing | Enhancing | Enhancing |
| | Granule 1 | Granule 2 | Granule 3 |
| Component | (mg/dose) | (mg/dose) | (mg/dose) |
| Crospovidone, NF | 17.50 | NA | 21.00 |
| (Polyplasdone XL) | | | |
| Polyethylene oxide, NF | 31.53 | 57.84 | 37.83 |
| (Polyox ™) | | | |
| Hypromellose, (Benecel K | 5.88 | 7.06 | 7.06 |
| 200M Pharm) | | | |
| Kollidon SR | 2.94 | 3.53 | 3.53 |
| Vitamin E (dl-α- | 0.13 | 0.15 | 0.15 |
| tocopherol | | | |
| Triethyl Citrate, NF | 2.03 | 3.42 | 2.43 |
| Docusate sodium, NF | 1.25 | 1.50 | 1.50 |
| (85%) with sodium | | | |
| benzoate, NF (15%) | | | |
| Colloidal silicon dioxide, | 1.25 | NA | NA |
| NF (Cab-O-Sil M-5P) | | | |
| Aerosil 200 | NA | 1.50 | 1.50 |
| Total | 62.51 | 75.00 | 75.00 |

Seal Coat

| | | | |
|---|---|---|---|
| Hypromellose (Methocel | 11.12 | NA | NA |
| E5 Premium LV) | | | |
| Triethyl citrate, NF | 1.12 | NA | NA |
| Colloidal silicon dioxide, | 0.25 | NA | NA |
| NF (Cab-O-Sil M-5P) | | | |
| Total | 75.00 | 75.00 | 75.00 |

### Manufacturing Procedure:

1. Polyox® was added to hypromellose, Kollidon® SR, docusate sodium, and crospovidone/starch 1500 in a high shear granulator and mixed to achieve a uniform powder mix using impeller and chopper.
2. A solution of dl-α-tocopherol solution and triethyl citrate was sprayed onto the powder mix from step #1 to achieve a uniform blend.
3. Colloidal silicon dioxide/Aerosil 200 was added to the blend from step #2 and mixed to achieve a uniform blend using an impeller and chopper.
4. The blend from step #3 was granulated by hot melt extrusion.
5. The granules from step #4 were processed using cryomilling to a mean particle size of 500 μm.

### Seal Coating Procedure:

1. Hypromellose was added to dehydrated alcohol in a stainless steel container and mixed to form a uniform dispersion.
2. To the dispersion from step #1, the purified water was added and mixed until a clear solution formed.
3. To the solution from step #2, triethyl citrate was added followed by the addition of colloidal silicon dioxide and mixed to form a homogenous dispersion.
4. The granules were coated using a Wurster fluid bed coater with an inlet air temperature of 40°-50° C., and sufficient air volume for fluidization.
5. When the product temperature reached 30° C., the dispersion from step #3 was sprayed onto the granules while maintaining the product temperature of 28°-30° C. and sufficient air volume for the fluidization, until the target coating weight gain (12.49 mg) was achieved.
6. The coated granules from step #5 were dried.

### Example 23: Tablet Composition

Oxycodone hydrochloride tablets (15 mg) are manufactured as described below:

TABLE 19
Formulation Composition of Oxycodone Hydrochloride Tablets,

| Components | mg/dose |
|---|---|
| Over coated active granules (Granule 3) | 286.24 |
| Viscosity enhancing granules (Granule 1) | 75.00 |
| Triggering granules (Granule 1) | 164.21 |
| Mannitol | 30.00 |
| Microcrystalline cellulose | 213.75 |
| Hydroxypropyl cellulose | 7.50 |
| Croscarmellose sodium | 18.75 |
| Magnesium stearate | 3.75 |
| Total | 799.20 |

### Manufacturing Procedure:

1. A uniform blend of over coated active granules, viscosity enhancing granules, triggering granules, anhydrous dibasic calcium phosphate, colloidal silicon dioxide, and croscarmellose sodium is made using a V-blender.
2. To the blend from step #1, magnesium stearate is added and blended for three minutes using a V-blender.
3. The blend from step #2 is compressed into tablets using a tablet press.

### Example 24: Tablet Composition

Hydromorphone hydrochloride tablets (8 mg) were manufactured as described below:

TABLE 20
Formulation Composition of Hydromorphone Hydrochloride Tablets,

| Components | mg/dose |
|---|---|
| Over coated active granules | 143.52 |
| Viscosity enhancing granules (1) | 75.00 |
| Triggering granules (1) | 164.23 |
| Microcrystalline cellulose | 262.25 |
| Mannitol | 30.00 |
| Hydroxypropyl cellulose | 7.50 |
| Croscarmellose sodium | 18.75 |
| Magnesium stearate | 3.75 |
| Total | 705.00 |

### Manufacturing Procedure:

1. A uniform blend of over coated active granules, viscosity enhancing granules, triggering granules, microcrystalline cellulose, mannitol, hydroxypropyl cellulose, and croscarmellose sodium was made using a V-blender.
2. To the blend from step #1, magnesium stearate was added and blended for three minutes using a V-blender.
3. The blend from step #2 was compressed into tablets using a tablet press.

### Example 25: Tablet Composition

Hydrocodone bitartrate tablets (10 mg) are manufactured as described below:

TABLE 21
Formulation composition of Hydrocodone Bitartrate Tablets,

| Components | mg/dose |
|---|---|
| Over coated active granules | 287.04 |
| Viscosity enhancing granules (1) | 75.00 |
| Triggering granules (2) | 121.65 |
| Microcrystalline cellulose | 93.81 |
| Croscarmellose sodium | 15.00 |
| Magnesium stearate | 7.50 |
| Total | 600.00 |

### Manufacturing Procedure:

1. A uniform blend of over coated active granules, viscosity enhancing granules, triggering granules, microcrystalline cellulose, and croscarmellose sodium is made using a V-blender.
2. To the blend from step #1, magnesium stearate is added and blended for three minutes using a V-blender.
3. The blend from step #2 is compressed into tablets using a tablet press.

### Example 26: Tablet Composition

Oxymorphone hydrochloride tablets are manufactured as described below:

TABLE 22
Formulation Composition of Oxycodone Hydrochloride Tablets

| Components | mg/dose |
|---|---|
| Over coated active granules | 143.52 |
| Viscosity enhancing granules(1) | 75.00 |
| Triggering granules(1) | 164.23 |
| Microcrystalline cellulose | 262.25 |
| Mannitol | 30.00 |
| Hydroxypropyl cellulose | 7.50 |
| Croscarmellose sodium | 18.75 |
| Magnesium stearate | 3.75 |
| Total | 705.00 |

### Manufacturing Procedure:

1. A uniform blend of over coated active granules, viscosity enhancing granules, triggering granules, microcrystalline cellulose, mannitol, hydroxypropyl cellulose, and croscarmellose sodium is made using a V-blender.
2. To the blend from step #1, magnesium stearate is added and blended for three minutes using a V-blender.
3. The blend from step #2 is compressed into tablets using a tablet press.

### Example 27: Opioid (10 mg) Capsule Dosage Form

Capsules filled with coated Opioid Particulates and Triggering Particulates.

TABLE 23
Formulation composition of oxycodone HCl (10 mg)
capsule dosage form

| Components | mg/dose |
|---|---|
| Opioid particulates (e.g., oxycodone | 100.00 |
| hydrochloride) | |
| Triggering particulates (magnesium | 220.00 |
| hydroxide granules) | |
| Total | 320.00 |

### Manufacturing Procedure:

1. A uniform blend of coated opioid particulates, and triggering particulates was made using a V-blender.
2. Based on the fill weight, the blend from Step #1 was filled into capsules.

### Example 28: Opioid (10 mg) Capsule Dosage Form

Coated Opioid Particulates were compressed into tablets, and filled into capsules along with Triggering Particulates.

TABLE 24
Formulation composition of oxycodone hydrochloride (10 mg)
capsule dosage form

| Components | mg/dose |
|---|---|
| Coated opioid particulates (e.g., oxycodone hydrochloride) | 100.00 |
| Microcrystalline cellulose | 14.5 |
| Anhydrous lactose | 14.5 |
| Hydroxypropyl cellulose | 34.00 |
| Croscarmellose sodium | 13.6 |
| Magnesium stearate | 3.40 |

External blend

| | |
|---|---|
| Triggering Particulates (magnesium hydroxide granules) | 220.00 |
| Total | 400.00 |

### Manufacturing Procedure:

1. A uniform blend of coated Opioid particulates, microcrystalline cellulose, anhydrous lactose, hydroxypropyl cellulose, and croscarmellose sodium was made using a V-blender.
2. To the blend from step #1, magnesium stearate was added and the mixture was further blended for 3 minutes.
3. The blend from step #2 was compressed into tablets using a tablet press.
4. The compressed tablets along with the triggering particulates were filled into capsules.

### Example 29: Opioid (10 mg) Bilayer Tablet Dosage Form

Coated opioid particulates and triggering particulates were compressed into bilayer tablets.

TABLE 25
Formulation composition of oxycodone hydrochloride (10 mg) or
hydrocodone bitartrate (10 mg) bilayer tablet dosage form

mg/dose

Active Tablet Components

| | |
|---|---|
| Coated opioid particulates (e.g., oxycodone or hydrocodone) | 100.00 |
| Microcrystalline cellulose | 14.50 |
| Anhydrous lactose | 14.50 |
| Hydroxypropyl cellulose | 34.00 |
| Croscarmellose sodium | 13.60 |
| Magnesium stearate | 3.40 |

Triggering Tablet Components

| | |
|---|---|
| Triggering Particulates (magnesium hydroxide granules) | 220.00 |
| Croscarmellose sodium | 4.75 |
| Magnesium stearate | 1.25 |
| Total | 406.00 |

### Manufacturing Procedure:

1. A uniform blend of coated Opioid Particulates, microcrystalline cellulose, anhydrous lactose, hydroxypropyl cellulose, and croscarmellose sodium was made using a V-blender.
2. To the blend from step #1, magnesium stearate was added and the mixture was further blended for 3 minutes using V-blender.
3. Similarly, a uniform blend of Triggering Particulates was made by mixing magnesium hydroxide granules and croscarmellose sodium using a V-blender.
4. To the blend from step #3, magnesium stearate was added and the mixture was further blended for 3 minutes using a V-blender.
5. The two blends (i.e., from step #2 and step #4) were layered on each other during compression to form bilayer tablets.

### Example 30: Opioid (10 mg) Capsule Dosage Form

Coated opioid particulates were compressed into a first tablet population. Triggering particulates were compressed into a second tablet population. The two tablet populations were filled into capsules.

TABLE 26
Formulation composition of oxycodone HCl (10 mg)
capsule dosage form

mg/dose

Active Tablet Components

| | |
|---|---|
| Coated Opioid Particulates (e.g., oxycodone hydrochloride) | 100.00 |
| Microcrystalline cellulose | 14.50 |
| Anhydrous lactose | 14.50 |
| Hydroxypropyl cellulose | 34.00 |
| Croscarmellose sodium | 13.60 |
| Magnesium stearate | 3.40 |

Triggering Tablet Components

| | |
|---|---|
| Triggering Particulates (1) | 220.00 |
| Croscarmellose sodium | 4.75 |
| Magnesium stearate | 1.25 |
| Total | 406.00 |

### Manufacturing Procedure:

1. A uniform blend of coated opioid particulates, microcrystalline cellulose, anhydrous lactose, hydroxypropyl cellulose, and croscarmellose sodium was made using a V-blender.
2. To the blend from step #1, magnesium stearate was added and blended for 3 minutes using a V-blender and then compressed into tablets using a tablet press.
3. Similarly, a uniform blend of triggering particulates was made by mixing magnesium hydroxide granules and croscarmellose sodium using a V-blender.
4. To the blend from step #3, magnesium stearate was added and the mixture was further blended for 3 minutes using V-blender and then compressed into tablets using a tablet press.
5. Tablets from step #2 and step #4 were filled into capsules.

### Example 31: In Vitro Overdose Protection (ODP) Studies with 60:40 Active Pellets

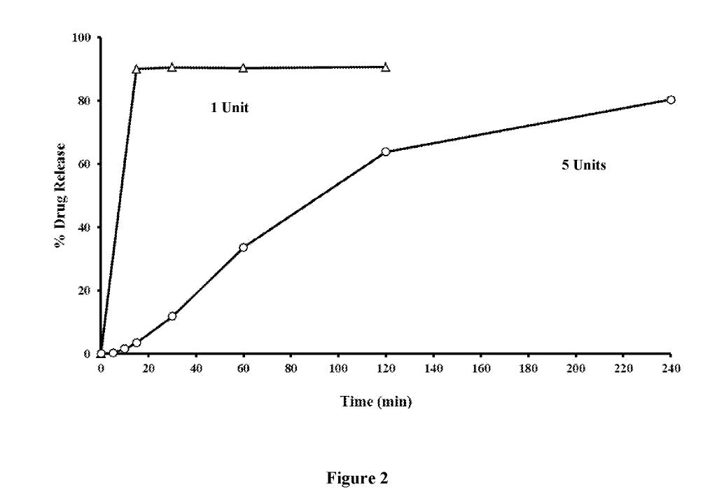

In order to examine the ability of the dosage form to prevent the release of its active when taken in doses above therapeutically effective amounts (e.g., three or more dosage units), taken in a manner inconsistent with the manufacturer's instructions, in a manner not prescribed, or overdosed, an in vitro dissolution test was conducted using a USP Apparatus II at pH 1.6. A pH of 1.6 was chosen to simulate the acidic environment of the stomach. FIG. 2 shows the percent release of oxycodone from the dosage form, wherein each unit represents a 30 mg oxycodone hydrochloride dosage form containing functional coated active pellets (Active Pellets 1) and Triggering Granules. In this Example, a functional coating with a ratio of OPADRY® cellulose acetate to EUDRAGIT® E PO of 60:40 was used.

### Experimental Procedure:

1. For each unit, 412.60 mg of functional coated Active Pellets 1 were combined with 350.00 mg of Triggering Granules 2 and placed in a capsule.
2. The capsule from step #1 was added to 250 mL of dissolution medium adjusted to a pH of 1.6.
3. Samples were withdrawn at 5, 10, 15, 30, 60, and 120 minutes for the single unit study and at 5, 10, 15, 30, 60, 120, and 240 minutes for the five unit study.
4. The samples obtained from step #3 were analyzed for the percent release of oxycodone by HPLC.

### Example 32: In Vitro Overdose Protection (ODP) Studies with 80:20 Active Pellets

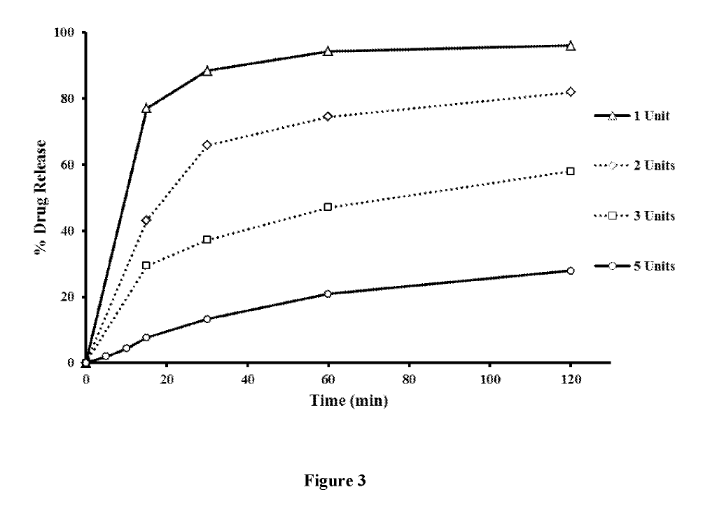

In order to examine the ability of the dosage form to prevent the release of its active when taken in doses above therapeutically effective amounts (e.g., three or more dosage units), taken in a manner inconsistent with the manufacturer's instructions, in a manner not prescribed, or overdosed, an in vitro dissolution test was conducted using a USP Apparatus II at pH 1.6. A pH of 1.6 was chosen to simulate the acidic environment of the stomach. FIG. 3 shows the percent release of oxycodone from the dosage form, wherein each unit represents a 30 mg oxycodone hydrochloride dosage form containing functional coated active pellets (Active Pellets 2) and Triggering Granules. In this Example, a functional coating with a ratio of OPADRY® cellulose acetate to EUDRAGIT® E PO of 80:20 was used.

As shown in FIGS. 2 and 3, the 80:20 functional coat was more effective than the 60:40 functional coat for oxycodone hydrochloride in this experimental model. The data suggest that a ratio of OPADRY® cellulose acetate to EUDRAGIT® E PO of 80:20 in the functional coat provided significantly superior ODP properties to a dosage form containing an active agent, e.g., oxycodone hydrochloride.

### Experimental Procedure:

1. For each unit, 412.60 mg of Functional Coated Active Pellets 2 was combined with 350.00 mg of Triggering Granules 2 and placed in a capsule.
2. The combination from step #1 was added to 250 mL of dissolution medium adjusted to a pH of 1.6.
3. Samples were withdrawn at 5, 10, 15, 30, 60, and 120 minutes for the single unit, two unit, three unit, and five unit studies.
4. The samples obtained from step #3 were analyzed for the percent release of oxycodone by HPLC.

### Example 33: In Vitro Overdose Protection (ODP) Studies with Opioid Formulation Containing 15 mg of Oxycodone Hydrochloride

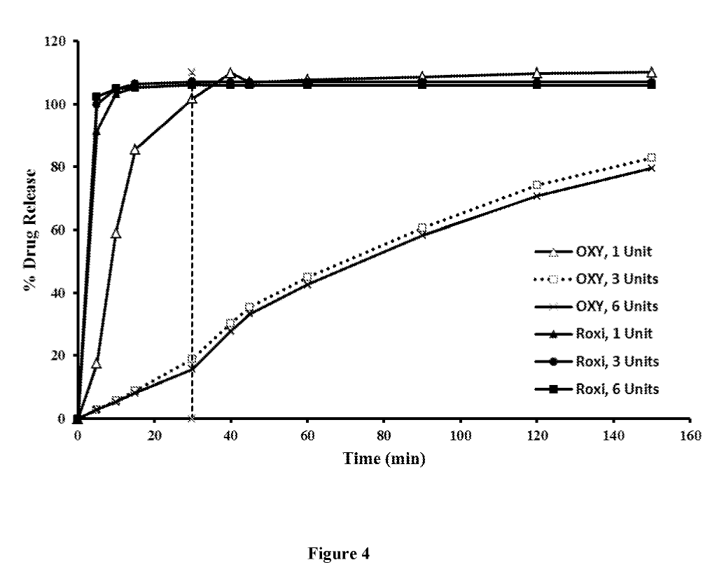

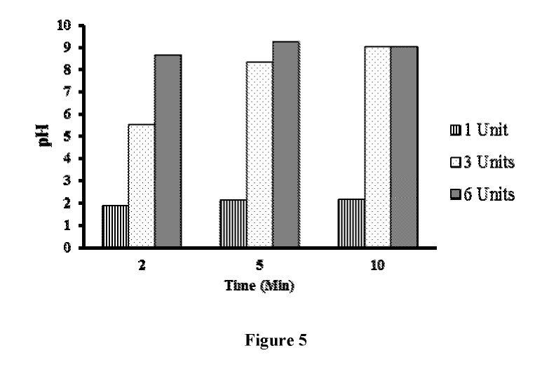

In order to examine the ability of the dosage form to prevent the release of its active when taken in doses above therapeutically effective amounts (e.g., three or more dosage units), taken in a manner inconsistent with the manufacturer's instructions, in a manner not prescribed, or overdosed, an in vitro dissolution test was conducted using a USP Apparatus II at pH 1.6 for 30 minutes followed by pH 6.8 for 120 minutes. In order to mimic physiological conditions, the total volume of the dissolution medium was kept at 250 ml at pH 1.6 acid medium, and 300 ml at pH 6.8. FIG. 4 shows dissolution profiles (% drug release) of oxycodone hydrochloride for 1, 3, and 6 oxycodone tablets (i.e., tablets of the invention; “OXY”; 15 mg), and for 1, 3, and 6 ROXICODONE tablets (“Roxi”; 15 mg). FIG. 5 shows the pH of the initial dissolution medium at 2, 5, and 10 minutes after adding 1, 3, or 6 oxycodone tablets of the invention.
1. Oxycodone hydrochloride tablet (15 mg) (Active granule 3, Triggering granule 1, and Viscosity enhancing granule 1), or ROXICODONE tablet, was added to a 250 ml acid-adjusted dissolution medium at pH 1.6, and the dissolution of the tablet was measured for 30 minutes.
2. 50 mL of 60 mM phosphate buffer was added to the solution from step #1, and the dissolution of the tablet was measured for an additional 120 minutes.
3. Samples were withdrawn from the solutions of steps #1 and #2 at intervals as shown in FIG. 4.
4. The samples obtained from step #3 were analyzed, using HPLC, for the percent release of oxycodone.
5. pH of the dissolution medium from step #1 (experiments with the oxycodone hydrochloride tablets of the invention) was measured at 2 minutes, 5 minutes, and 10 minutes after introduction of the tablet(s).
6. Steps #1-5 were repeated for addition of 3 and 6 dosage units (3 and 6 tablets).

The results showed that a single tablet had no appreciable effect on variation of pH with time (at 2, 5, and 10 minutes); however, with multiple tablets (3 and 6 tablets), the pH was greater than 5 within 2 minutes (FIG. 5). The rapid rise in pH with multiple tablets can be attributed to the amount of pH modifier present in the pH triggering granules, and the rapid disintegration of the tablet. As a result of the rise in pH above 5 within 2 minutes, the pH-dependent polymer EUDRAGIT®E PO, which acts as a pore former in the functional coating, becomes insoluble, thus changing the release mechanism from pore-mediated transport to true diffusion.

### Example 34: In Vitro Overdose Protection (ODP) Studies with Opioid Formulation Containing 8 mg of Hydromorphone Hydrochloride

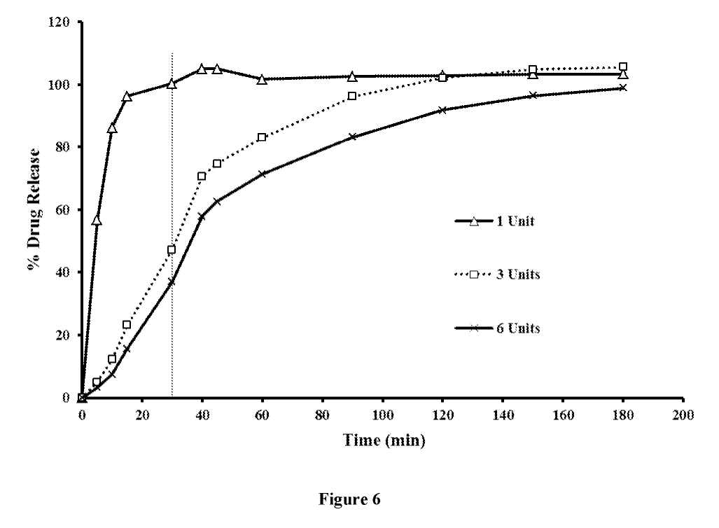

In order to examine the ability of the dosage form to prevent the release of its active when taken in doses above therapeutically effective amounts (e.g., three or more dosage units), taken in a manner inconsistent with the manufacturer's instructions, in a manner not prescribed, or overdosed, an in vitro dissolution test was conducted using a USP Apparatus II at pH 1.6 acid medium for 30 minutes followed by pH 6.8 for 150 minutes. In order to mimic physiological conditions, the total volume of the dissolution medium was kept at 250 ml at pH 1.6, and 300 ml at pH 6.8. FIG. 6 shows dissolution profiles (% drug release) of hydromorphone hydrochloride for 1, 3, and 6 hydromorphone tablets (i.e., tablets of the invention; 8 mg).
1. Hydromorphone hydrochloride tablet (8 mg) (Active Granules, Triggering Granules 1, and Viscosity Enhancing Granules 1) was added to a 250 ml acid-adjusted dissolution medium at pH 1.6, and the dissolution of the tablet was measured for 30 minutes.
2. 50 ml of 60 mM phosphate buffer was added to the solution from step #1, and dissolution of the tablet was measured for additional 150 minutes.
3. The samples were withdrawn from the solutions of step #1 and #2, at intervals as shown in FIG. 6.
4. The samples obtained from step #3 were analyzed for the percent release of hydromorphone hydrochloride by HPLC.
5. Steps #1-4 were repeated for 3 and 6 dosage units (3 and 6 tablets).

### Example 35: In Vitro Abuse Deterrent Studies (Resistance to Grindability)

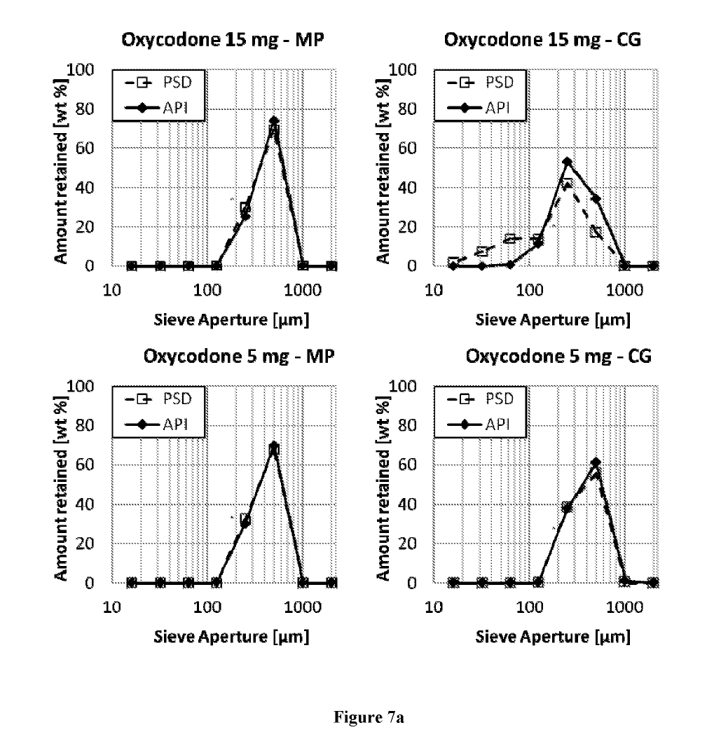

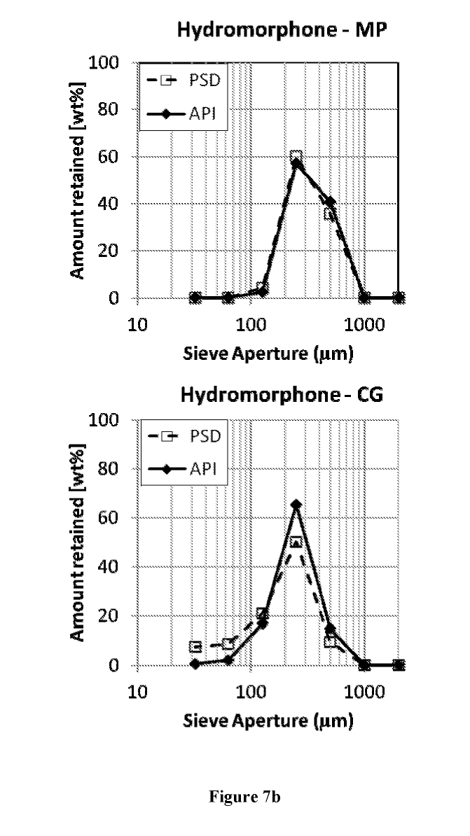

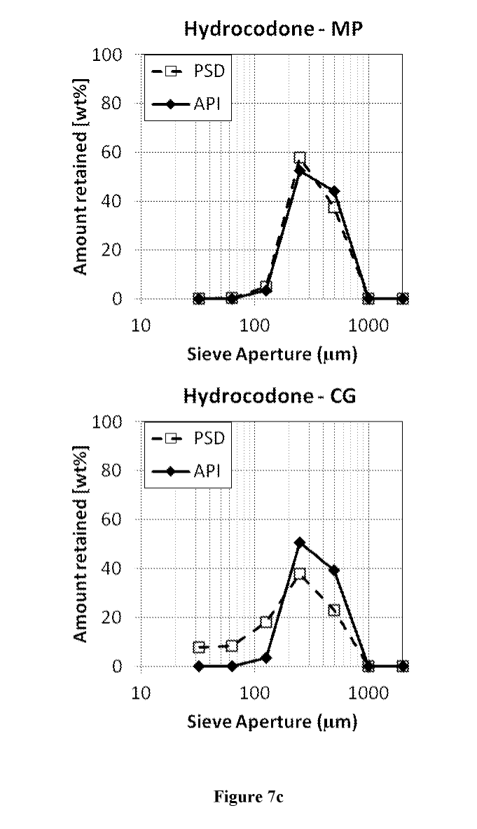

In order to examine the abuse resistance (e.g., ability to withstand grinding) of Active Granules, an in vitro physical manipulation test was conducted for various opioids, e.g., oxycodone, hydromorphone, and hydrocodone. FIGS. 7a-c show the results of particle size distribution (PSD) and API distribution from manipulated (by mortar and pestle (MP) or by electric coffee grinder (CG)) active granules of oxycodone hydrochloride, hydromorphone hydrochloride, and hydrocodone bitartrate respectively, across sieve fractions. In general, the API distribution follows PSD across sieve fractions as API stayed “locked-in” with the granules. FIGS. 7a-c demonstrate the nongrindable and noncrushable nature of Active Granules. The data demonstrate that even after grinding, the weight % of fine particles (i.e., particle size of below 125 μm; “fines fraction”) remains very low, thereby inhibiting or preventing the abuser from snorting the active agent, even after tampering with the dosage form by grinding.

The results corroborate that the opioid granules have crush resistant properties and the majority of granules produced after grinding are in the size range of 250-500 μm. Simply from the size perspective, these granules are harder to snort compared to fine powder with a particle size of less than 250 μm. Furthermore, the majority of API resides with the larger granules, thereby reducing the effective amount of drug that can be snorted. If an abuser is still able to snort the particles, the dissolution rate of the API will be much slower due to the pH-sensitive coating and the viscosity enhancing polymer, thus drastically lowering the effective amount of drug delivered to the abuser (and required to achieve euphoria).

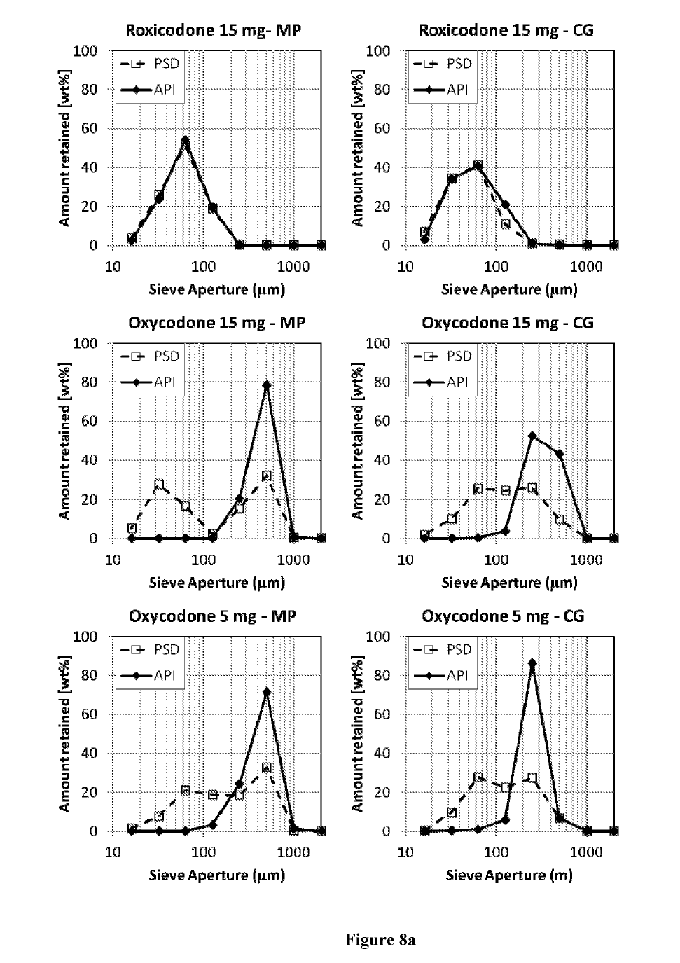

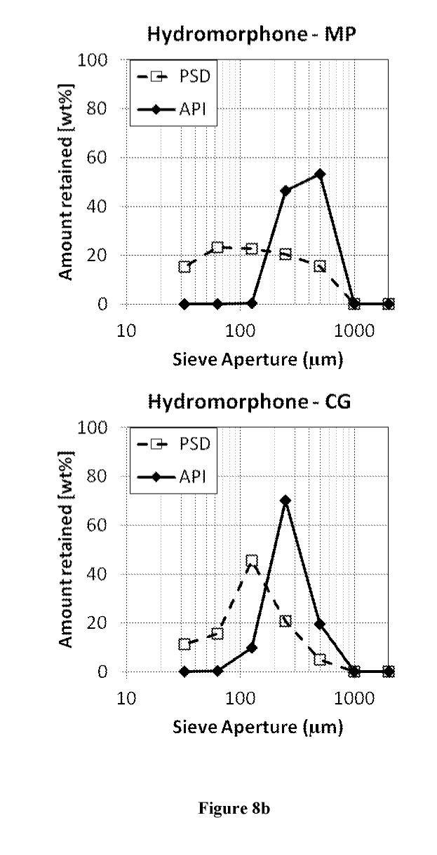

FIGS. 8a-b show the results of particle size distribution and API distribution from manipulated tablets of oxycodone hydrochloride (FIG. 8a) and hydromorphone hydrochloride (FIG. 8b) (i.e., tablets of the invention), as well as manipulated tablets of ROXICODONE (FIG. 8a), across sieve fractions. FIG. 8a compares particle size distribution and API distribution of oxycodone hydrochloride tablets and ROXICODONE tablets. The data show 100% of ROXICODONE particulates were in the size range from about 30-125 μm. Further, the API distribution is superimposed with the particle size distribution, suggesting that the particles, and the API contained within, were not resistant to size reduction. In contrast, there are broad particle size distributions, from about 16-500 μm, for oxycodone tablets of the invention (15 mg and 5 mg). The API distribution for oxycodone tablets is skewed towards the right, i.e., more API is present in granules with particle sizes of about 250-500 μm, suggesting that API remained “locked-in” the granules of the invention, which resist size reduction. Despite the large size, if an abuser succeeds in insufflating/inhaling the resulting granular remnants of the manipulated tablets, drug release will be further compromised because of the low fluid volume in the nasal mucosa and the pH of nasal fluids. Essentially 100% of the API remained “locked-in” the granules that are difficult to snort, e.g., granules in the size range of 250-500 μm.

### Grinding Procedure for Opioid Granules:

1. Four grams of opioid (e.g., oxycodone hydrochloride, hydromorphone hydrochloride, and hydrocodone bitartrate granules) granules were crushed in a Mortar and Pestle for 5 minutes or ground in a Hamilton Beach Coffee Grinder (Model #80365) for 2 minutes.
2. The powder was analyzed by sieve analysis using the following mesh sizes: 10 (2000 μm), 18 (1000 μm), 35 (500 μm), 60 (250 μm), 120 (125 μm), 230 (63 μm), and 425 (32 μm).
3. API distribution across all sieve fractions was determined by analyzing the API content in each sieve fraction by HPLC method using external reference standard.

### Grinding Procedure for Oxymorphone Granules:

5. Four grams of oxymorphone granules are crushed in a Mortar and Pestle for 5 minutes or ground in a Hamilton Beach Coffee Grinder (model 80365) for 2 minutes.
6. The powder is analyzed by sieve analysis using the following mesh sizes: 0 (2000 μm), 18 (1000 μm), 35 (500 μm), 60 (250 μm), 120 (125 μm), 230 (63 μm), and 425 (32 μm).
7. API distribution across all sieve fractions is determined by analyzing the API content in each sieve fraction by HPLC method using external reference standard.

### Grinding Procedure for Opioid Tablets:

1. Opioid tablets (oxycodone hydrochloride tablets and hydromorphone hydrochloride tablets of the invention, and ROXICODONE tablets) were crushed in a Mortar and Pestle for 5 minutes or ground in a Hamilton Beach Coffee Grinder (model 80365) for 2 minutes.
2. The powder was analyzed by sieve analysis using the following mesh sizes: 10 (2000 μm), 18 (1000 μm), 35 (500 μm), 60 (250 μm), 120 (125 μm), 230 (63 μm), and 425 (32 μm).
3. API distribution across all sieve fractions was determined by analyzing the API content in each sieve fraction by HPLC method using external reference standard.

### Grinding Procedure for Oxymorphone Tablets:

1. Oxymorphone tablets are crushed in a Mortar and Pestle for 5 minutes or ground in a Hamilton Beach Coffee Grinder (model 80365) for 2 minutes.
2. The powder is analyzed by sieve analysis using the following mesh sizes: 10 (2000 μm), 18 (1000 μm), 35 (500 μm), 60 (250 μm), 120 (125 μm), 230 (63 μm), and 425 (32 μm).
3. API distribution across all sieve fractions is determined by analyzing the API content in each sieve fraction by HPLC method using external reference standard.

### Example 36: In Vitro Abuse Deterrent Studies (Resistance to Extractability and Syringeability)

1. One tablet of opioid (e.g., oxycodone hydrochloride or hydromorphone hydrochloride) was crushed in a mortar and pestle for 5 minutes.
2. To the crushed tablet from step #1, 10 ml of water (at ambient temperature) was added to form a mixture.
3. The mixture from step #2 was vortexed for 15 seconds and maintained at ambient temperature, e.g., 25° C., for 30 minutes with occasional stirring.
4. The supernatant liquid from the mixture from step #3 was withdrawn through an 18 gauge needle into a 10 ml syringe while recording the time for withdrawal and the volume withdrawn.
5. The API content present in the withdrawn liquid was determined via HPLC analysis using an external reference standard.
6. Effort required to withdraw the liquid in step #4 was calculated as time needed to withdraw 1 ml of the liquid (time required to withdraw the liquid/total amount of liquid withdrawn).

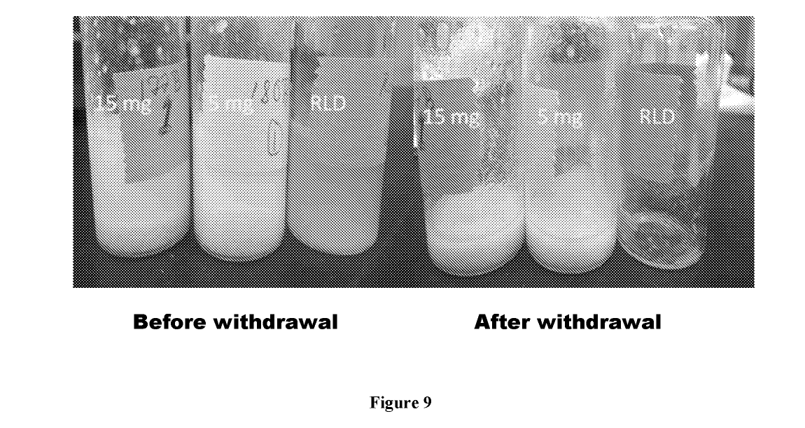

FIG. 9 compares the suspensions resulting from the dissolution of 5 mg and 15 mg of crushed oxycodone hydrochloride tablets (of the invention), and 15 mg of crushed ROXICODONE® tablets (RLD). Before withdrawal, the oxycodone hydrochloride products of the invention show two layers: a viscous gel layer at the bottom and a lightly turbid supernatant on the top, while ROXICODONE® (RLD) shows more uniform lightly turbid suspension. The figure shows (After withdrawal) residual amounts of viscous liquid (15 mg and 5 mg tablets of the invention) left in the vials after the removal of supernatant liquid by the syringe. As shown, essentially all liquid can be syringed from the vial for crushed ROXICODONE® tablets, whereas a large portion of the bottom gel layer is not syringeable for crushed oxycodone hydrochloride tablets of the invention.

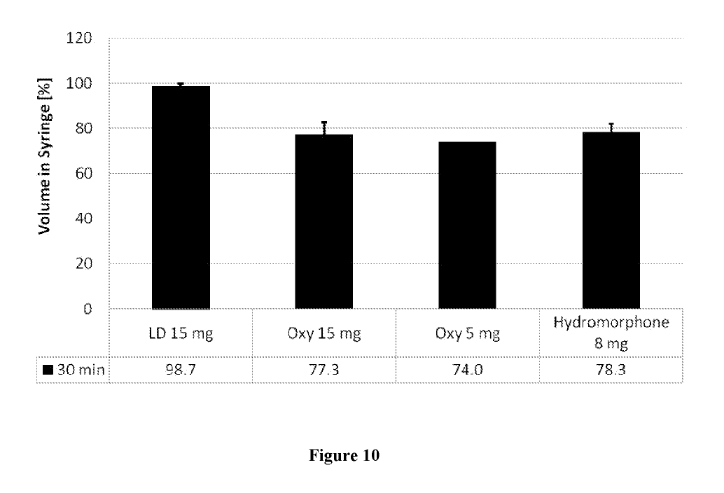

FIG. 10 shows percent volume of supernatant liquid withdrawn in a syringe. The data show that at 30 minutes of incubation in 10 ml water, almost 100% of the liquid is syringeable for ROXICODONE®, while only 70-80% of the supernatant liquid is syringeable for oxycodone hydrochloride and hydromorphone hydrochloride tablets of the invention.

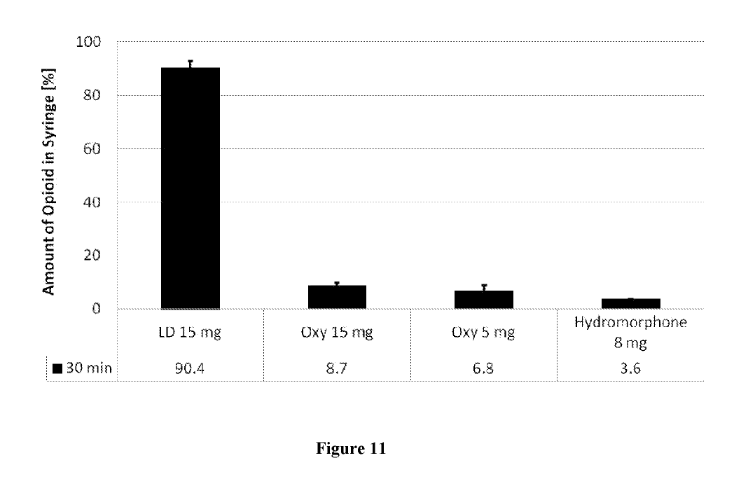

FIG. 11 shows the amount of API present in the withdrawn liquid. The data show that the withdrawn fluid from ROXICODONE® tablets contains 90% of API, compared to less than 10% API in the withdrawn fluid from oxycodone hydrochloride and hydromorphone hydrochloride tablets of the invention. Thus, despite 70-80% syringeability, the amount of API that can be extracted for intravenous abuse in tablets of the present invention is reduced substantially compared to ROXICODONE®. The combination of, at least, viscosity enhancing polymer and pH-sensitive coating significantly reduced the amount of API that could be extracted for intravenous abuse.

1-78. (canceled)

79. A solid oral immediate release multi-particulate dosage form with abuse deterrent and enhanced overdose protection properties comprising:
a first population of crush resistant Active Particulates comprising a therapeutically effective amount of an opioid embedded in a polymer matrix, and an acid labile functional coat comprising two functional coat layers over the polymer matrix;
wherein the two functional coat layers comprise functional coat layer 1 and functional coat layer 2, and
wherein functional coat layer 2 surrounds functional coat layer 1;
wherein functional coat layer 1 comprises a nonionic rate-controlling polymer insoluble in physiological fluids and/or organic solvents, and at least one cationic polymer, in a ratio of 80:20, and functional coat layer 2 comprises at least one cationic polymer and, optionally, a nonionic rate-controlling polymer; and
a second population of Triggering Particulates comprising an alkaline agent;
wherein the enhanced overdose protection properties comprise resistance to release of the opioid from the dosage form when three or more units of the dosage form are consumed intact, such that less than about 50% of the opioid is released at 30 minutes; and
wherein the presence of functional coat layer 2 further enhances the resistance to release of the opioid from the dosage form provided by functional coat layer 1.

80. The dosage form of claim 79, wherein the abuse deterrent properties comprise resistance to syringeability by limiting the extractability of the opioid whereby less than about 30% of the opioid is available in syringeable form, and resistance to grinding and crushing such that grinding or crushing of the first population of particulates provides more than 50% of particulates in the size range of 250-500 μm.

81. The dosage form of claim 80, wherein the syringeable form is a syringeable liquid obtained by adding at least one crushed dosage form to 10 ml of water at room temperature, forming a suspension, vortexing the suspension for about 15 seconds, and maintaining the suspension for about 30 minutes.

82. The dosage form of claim 81, wherein the syringeable liquid is withdrawn through an 18 gauge needle into a syringe.

83. The dosage form of claim 79, wherein the cationic polymer is a copolymer based on dimethylaminoethyl methacrylate, butyl methacrylate, and methyl methacrylate.

84. The dosage form of claim 79, wherein the polymer matrix comprises a nonionic polymer selected from the group consisting of a copolymer of ethyl acrylate, methyl methacrylate, and a low content of methacrylic acid ester with quaternary ammonium groups; hydroxypropyl cellulose; hydroxypropyl methylcellulose; hydroxyethylcellulose; ethylcellulose; cellulose acetate butyrate; cellulose acetate; polyvinyl acetate based polymers; and polyethylene oxide polymers.

85. The dosage form of claim 84, wherein the nonionic polymer is a mixture of a polyethylene oxide polymer and hydroxypropyl methylcellulose.

86. The dosage form of claim 79, wherein the alkaline agent present in the second population of Triggering Particulates is selected from the group consisting of aluminum hydroxide, sodium hydroxide, potassium hydroxide, calcium hydroxide, magnesium hydroxide, calcium carbonate, sodium carbonate, potassium bicarbonate, sodium bicarbonate, ammonia, tertiary sodium phosphate, diethanolamine, ethylenediamine, N-methylglucamine, L-lysine, and combinations thereof.

87. The dosage form of claim 86, wherein the alkaline agent is magnesium hydroxide.

88. The dosage form of claim 79, wherein the Triggering Particulates further comprise a pH-stabilizing agent selected from the group consisting of bismuth aluminate, bismuth carbonate, bismuth subcarbonate, bismuth subgallate, bismuth subnitrate, calcium phosphate, dibasic calcium phosphate, dihydroxyaluminum aminoacetate, dihydroxyaluminum, glycine, magnesium glycinate, sodium potassium tartrate, tribasic sodium phosphate, tricalcium phosphate, and combinations thereof.

89. The dosage form of claim 88, wherein the pH-stabilizing agent is dibasic calcium phosphate.

90. The dosage form of claim 79, wherein the polymer matrix of the first population of Active Particulates further comprises a plasticizer in an amount sufficient to enhance elasticity and crush resistance of the polymer matrix.

91. The dosage form of claim 90, wherein the plasticizer acts as an aversion agent and/or a tissue irritant.

92. The dosage form of claim 90, wherein the plasticizer is selected from the group consisting of triethyl citrate, propylene glycol, polyethylene glycols, triacetin, diethylene glycol monoethyl ether, dibutyl sebacate, and diethyl phthalate.

93. The dosage form of claim 79, wherein the first population of Active Particulates further comprises a surfactant.

94. The dosage form of claim 79, wherein the dosage form further comprises a third population of particulates comprising a viscosity-enhancing agent comprising a nonionic polymer and/or an anionic polymer.

95. The dosage form of claim 94, wherein the viscosity-enhancing agent is a mixture of the nonionic polymer and the anionic polymer.

96. The dosage form of claim 95, wherein the nonionic polymer is a polyethylene oxide polymer and the anionic polymer is a carbomer.

97. The dosage form of claim 94, wherein the viscosity-enhancing agent provides resistance to extraction of the opioid and withdrawal of extracted fluid into a syringe after attempting to dissolve one or more intact, crushed, or ground dosage units.

98. The dosage form of claim 79, wherein the particulates in the size range of 250-500 μm contain more than 75% of the opioid.

99. The dosage form of claim 79, wherein the opioid is selected from the group consisting of oxycodone, hydrocodone, oxymorphone, hydromorphone, and pharmaceutically acceptable salts thereof.

100. A solid oral immediate release multi-particulate dosage form with abuse deterrent and enhanced overdose protection properties comprising:
a first population of crush resistant Active Particulates comprising a therapeutically effective amount of an opioid embedded in a polymer matrix, and an acid labile functional coat comprising two functional coat layers over the polymer matrix;
wherein the two functional coat layers comprise functional coat layer 1 and functional coat layer 2, and wherein functional coat layer 2 surrounds functional coat layer 1;
wherein functional coat layer 1 comprises a nonionic rate-controlling polymer insoluble in physiological fluids and/or organic solvents, and at least one cationic polymer, in a ratio of 80:20, and functional coat layer 2 comprises at least one cationic polymer and, optionally, a nonionic rate-controlling polymer; and
a second population of Triggering Particulates comprising an alkaline agent; and
wherein the enhanced overdose protection properties comprise resistance to release of the opioid when three or more units of the dosage form are subjected to dissolution in a medium at pH 1.6 for 30 minutes, such that less than about 50% of the opioid is released at 30 minutes.

101. The dosage form of claim 99, wherein less than about 25% of the opioid is released at 30 minutes.

102. The dosage form of claim 99, wherein the pH of the dissolution medium is greater than about 5 within two minutes when three or more dosage units are dissolved.
